(19) United States
(12) Patent Application Publication
(10) Pub. No.: US 2024/0287534 A1
(43) Pub. Date: Aug. 29, 2024

(54) INTEGRATED MOLECULAR AND GLYCO-ENGINEERING OF COMPLEX VIRAL GLYCOPROTEINS

(71) Applicants: UNIVERSITY OF CAPE TOWN, Cape Town (ZA); UNIVERSITY OF NATURAL RESOURCES AND LIFE SCIENCES VIENNA (BOKU), Wien (AT)

(72) Inventors: Edward Peter Rybicki, Cape Town (ZA); Emmanuel Aubrey Margolin, Cape Town (ZA); Richard Strasser, Vienna (AT)

(73) Assignees: UNIVERSITY OF CAPE TOWN, Cape Town (ZA); UNIVERSITY OF NATURAL RESOURCES AND LIFE SCIENCES VIENNA (BOKU), Vienna (AT)

(21) Appl. No.: 18/289,937

(22) PCT Filed: May 10, 2022
(86) PCT No.: PCT/IB2022/054318

§ 371 Date: Nov. 8, 2023

(30) Foreign Application Priority Data
May 10, 2021 (GB) 2106659.2

(51) Int. Cl. C12N 15/82

(52) U.S. Cl. CPC C12N 15/8258

(57) ABSTRACT

This invention relates to a method for increasing the expression, increasing glycosylation efficiency, reducing plant specific modifications, reducing aggregation and/or promoting the correct folding and oligomerisation of a heterologous polypeptide of interest in a plant cell, preferably a complex glycoprotein, wherein the method comprises co-expressing the heterologous polypeptide of interest with (i) a polypeptide encoding a mammalian chaperone protein, (ii) a polypeptide which improves N-glycan occupancy in the heterologous polypeptide of interest, and (iii) a nucleic acid which interferes with an enzyme which is responsible for the formation of truncated glycans in the plant cell and thus reduces the formation of truncated glycans. The invention further relates to plant cells and plants which, either transiently or stably, co-express the heterologous polypeptide of interest, the mammalian chaperone protein, the polypeptide which improves glycan occupancy and nucleic acid.

### BACKGROUND OF THE INVENTION

The production of complex glycoproteins in plants, and in particular viral glycoproteins, poses a challenge due to low expression yields, non-native glycosylation and inefficient maturation (folding) of these proteins along the secretory pathway. The molecular basis for this has been unclear and this has severely hampered the widespread implementation of molecular farming as a viable pharmaceutical production system. Instead, the technology has mostly been confined to niche applications where mainstream industry has failed to satisfy market demands. Our previous work, and the work described here, demonstrates that this is due to differences in the host cellular machinery which do not support efficient glycosylation, chaperone-mediated folding and proteolytic processing, ultimately hindering the folding of these proteins. This constrains the production of complex glycosylated proteins in the system and precludes the use of plants to consistently produce vaccines from complex heavily glycosylated viral glycoproteins. This is similarly prohibitive for the production of other complex glycoprotein-based pharmaceuticals.

The production of complex glycoproteins proteins in plants often leads to low yields, inefficient processing (maturation/folding) and plant-specific glycosylation which does not adequately resemble the structure and glycosylation of the native protein. The plant-glycosylation machinery poses several challenges to the development of human pharmaceuticals, such as inefficient glycosylation which may lead to poor glycan occupancy, potentially immunogenic plant-specific modifications and other non-native glycan processing that results in glycoforms that are not present on mammalian glycoproteins. However, the prevalence of these glycoforms was not previously described for heavily glycosylated viral glycoproteins, and sufficient quantitative analyses for plant-produced glycoproteins are lacking in general. Therefore, it is not well understood how the plant glycosylation machinery impacts production of complex viral glycoproteins or other similarly complex glycosylated proteins. The inventors delineated their prevalence and highlighted that inefficient glycosylation in plants compromised protein folding. The inventors further addressed these constraints by integrating various glyco-engineering strategies with chaperone co-expression enabling the production of a recombinant HIV Env gp140 trimer which closely resembled the equivalent mammalian cell-produced protein. They subsequently applied these approaches to produce other similarly glycosylated viral glycoproteins in plants from prototype emerging viruses. This technology is broadly applicable and now enables the production of heavily glycosylated complex glycoproteins in plants that resemble the native protein. This approach enables the production of well-folded and appropriately glycosylated complex glycoproteins in plants for the first time, thereby facilitating the production of vaccines and therapeutics in plants that could not previously be produced. Furthermore, the glycans decorating plant-produced glycoproteins that are produced using this approach could also be further engineered to contain mammalian-type extensions including, but not limited to, α1,6-fucosylation, β1,4-galactosylation and α2,6-sialylation.

Few plant-produced glycoproteins have advanced to clinical testing. Medicago Inc., who are arguably the global leaders in molecular farming, have not addressed fundamental differences in glycosylation between naturally produced and plant produced proteins which likely preclude the production of many complex proteins in their native state. Their technology platform has successfully resulted in influenza and SARS-CoV-2 VLP vaccines which have been tested in clinical trials. However, these antigens do not fully recapitulate the structure of the native glycoproteins and their technology platform does not address critical host constraints that are necessary to produce other complex glycoproteins. Their vaccines also contain plant-specific glycans and it is unclear if they are well-glycosylated. Their platform requires fusion of the protein of interest to the transmembrane and cytoplasmic tails of influenza to stabilize the trimer and generate VLPs. Whilst this is highly effective for SARS-CoV-2 it may compromise the native structure of other viral antigens which could be important for appropriate immunogenicity. Our work provides an integrated approach to produce complex viral (and other) glycoproteins in plants that recapitulate important structural features and critical elements of their glycosylation which are required for appropriate immunogenicity. The work also forms a basis to produce glycoproteins with tailor-made glycosylation to improve potency of therapeutics and efficacy of vaccines. This is similarly applicable to other viral glycoproteins, such as antibodies, which have value as therapeutics.

### SUMMARY OF THE INVENTION

The present invention relates to methods for increasing the expression, increasing glycosylation efficiency, reducing plant specific modifications, reducing aggregation and/or promoting the correct folding and oligomer assembly of heterologous polypeptides of interest in a plant cell. Preferably, the heterologous polypeptides are complex glycoproteins. The method comprises the steps of co-expressing the heterologous polypeptide of interest with (i) a polypeptide encoding a mammalian chaperone protein, (ii) a polypeptide which improves N-glycan occupancy in the heterologous polypeptide of interest, and (iii) a nucleic acid which interferes with an enzyme which is responsible for the formation of truncated glycans in the plant cell and which reduces the formation of truncated glycans. The invention also relates to plant cells and plants which, either transiently or stably, co-express the heterologous polypeptide of interest, the mammalian chaperone protein, the polypeptide which improves glycan occupancy and the nucleic acid.

In a first aspect of the invention there is provided for a method for producing heterologous polypeptides of interest in a plant cell. It will be appreciated that the heterologous polypeptides of interest may be a glycoprotein, preferably the glycoprotein is for use in pharmaceutical applications, vaccines, diagnostics, therapeutics and/or research reagents. It will also be appreciated that the polypeptide of interest may be for use in either humans or animals. The method comprising or consisting of firstly providing a first nucleic acid which encoding a mammalian chaperone protein, providing a second nucleic acid encoding a polypeptide which increases glycan occupancy, specifically wherein the second polypeptide increases glycosylation efficiency, more specifically N-glycosylation efficiency, providing a third nucleic acid which interferes with an enzyme which is responsible for the formation of truncated glycans in the plant cell, and providing a fourth nucleic acid encoding a heterologous polypeptide of interest. Secondly, cloning the first, second, third and fourth nucleic acids into at least one expression vector adapted to express a polypeptide in a plant cell and transforming or infiltrating a plant cell with the at least one expression vector of step. Thirdly, co-expressing the polypeptide encoding the mammalian chaperone protein, the polypeptide which increases glycan occupancy, the nucleic acid which interferes with the enzyme responsible for the formation of truncated glycans and the heterologous polypeptide of interest in the plant cell, and finally recovering the heterologous polypeptide of interest from the plant cell.

In one embodiment of the invention the method results in at least one or more of the following: (i) increased expression of the heterologous polypeptide of interest; (ii) increased glycosylation efficiency of the heterologous polypeptide of interest; (iii) a reduction in plant specific modifications of the heterologous polypeptide of interest; (iv) a reduction in aggregation of the heterologous polypeptide of interest; (v) increased folding efficiency of the heterologous polypeptide of interest; and/or (vi) improved oligomerisation of the heterologous polypeptide of interest.

In a second embodiment of the invention the chaperone protein is a mammalian chaperone protein, preferably the mammalian chaperone protein is at least one human chaperone protein selected from the group consisting of calnexin, calreticulin, GRP78/BiP, GRP94, GRP170, HSP47, ERp29, protein disulfide isomerase, peptidyl prolyl cis-trans-isomerase and ERp57. More preferably, the human chaperone protein is selected from calnexin and/or calreticulin.

In a third embodiment of the invention the polypeptide which increases glycan occupancy is an oligosaccharyltransferase enzyme. Preferably, the oligosaccharyltransferase enzyme is LmSTT3D from Leishmania major. Although those of skill in the art will appreciate that any protein which increases glycan occupancy in the heterologous polypeptide of interest will result in more efficient glycosylation of the heterologous polypeptide of interest.

In a fourth embodiment of the invention there is provided for a third nucleic acid which is an is an RNAi expression cassette encoding an RNAi agent which interferes with a protein which is responsible for producing paucimannosidic/truncated glycans produced in the cell. Preferably the RNAi agent interferes with a protein expressed from the hexosaminidase 3 gene. Even more preferably the RNAi agent reduces the expression of the hexosaminidase 3 protein in the cell, thereby reducing the amount of paucimannosidic/truncated glycans produced in the cell.

In a fifth embodiment of the invention the plant cell is a Nicotiana benthamiana cell. Preferably, the N. benthamiana cell is a glycosylation mutant lacking plant-specific N-glycan residues.

In a sixth embodiment of the invention the heterologous polypeptide of interest is a glycoprotein. Preferably the glycoprotein is a viral glycoprotein is for use in pharmaceutical applications, vaccines, diagnostics, therapeutics and/or research reagents.

In a further embodiment of the invention the at least one expression vector includes promoters and/or other regulators, operably linked to the first, second, third and fourth nucleic acids. It will be appreciated that the first, second, third and fourth nucleic acids may be contained on one, two, three or four expression vectors. Further, if the invention comprises one expression vector then the first, second, third and fourth nucleic acids are contained on that vector. If the invention comprises two expression vectors then the first, second, third and fourth nucleic acids may be contained on the two expression vectors in any combination of one nucleic acid on the first vector and three nucleic acids on the second vector or in any combination of two nucleic acids on the first vector and two nucleic acids on the second vector, provided that each of the first, second, third and fourth nucleic acids are all present. It will further be appreciated that if the invention comprises three vectors then the first, second, third and fourth nucleic acids may be contained on the three expression vectors in any combination of one nucleic acid on the first vector, one nucleic acid on the second vector and two nucleic acids on the third vector, provided that each of the first, second, third and fourth nucleic acids are all present. Alternatively, the invention may comprise four expression vectors wherein each of the first, second, third and fourth nucleic acids is contained on its own vector.

In a second aspect of the invention there is provided for a plant cell which is transformed with at least one expression vector, comprising or consisting of a first nucleic acid encoding a mammalian chaperone protein, a second nucleic acid encoding a polypeptide which increases glycan occupancy, a third nucleic acid which interferes with an enzyme which is responsible for the formation of truncated glycans in the plant cell, and a fourth nucleic acid encoding a heterologous polypeptide of interest. It will be appreciated that the aforementioned nucleic acids may be contained on one, two, three or four expression vectors.

In a first embodiment of the second aspect the chaperone protein is a mammalian chaperone protein, preferably the mammalian chaperone protein is a human chaperone protein selected from the group consisting of calnexin, calreticulin, GRP78/BiP, GRP94, GRP170, HSP47, ERp29, protein disulfide isomerase, peptidyl prolyl cis-trans-isomerase and ERp57. More preferably, the human chaperone protein is selected from calnexin and/or calreticulin

In a second embodiment of the second aspect of the invention the polypeptide which increases glycan occupancy is an oligosaccharyltransferase enzyme. Preferably, the oligosaccharyltransferase enzyme is LmSTT3D from Leishmania major. Those of skill in the art will appreciate that any protein which increases glycan occupancy in the heterologous polypeptide of interest will result in more efficient glycosylation of the heterologous polypeptide of interest.

In a third embodiment of the second aspect of the invention there is provided for a third nucleic acid which is an is an RNAi expression cassette encoding an RNAi agent which interferes with a protein which is responsible for producing paucimannosidic/truncated glycans produced in the cell. Preferably the RNAi agent interferes with a protein expressed from the hexosaminidase 3 gene. Even more preferably the RNAi agent reduces the expression of the hexosaminidase 3 protein in the cell, thereby reducing the amount of paucimannosidic/truncated glycans produced in the cell.

In a fourth embodiment of the second aspect of the invention the heterologous polypeptide of interest is a glycoprotein. Preferably the glycoprotein is a viral glycoprotein is for use in pharmaceutical applications, vaccines, diagnostics, therapeutics and/or research reagents.

In a fifth embodiment of the second aspect of the invention the at least one expression vector includes promoters and/or other regulators, operably linked to the first, second, third and fourth nucleic acids. As mentioned hereinbefore the first, second, third and fourth nucleic acids may be present in the cell on one, two, three or four expression vectors.

In a sixth embodiment of the second aspect of the invention it will be appreciated that the plant cell may be from either a monocotyledonous or dicotyledonous plant. Preferably, the plant cell is from a plant selected from the group consisting of maize, rice, sorghum, wheat, cassava, barley, oats, rye, sweet potato, soybean, alfalfa, tobacco, sunflower, cotton, and canola. More preferably, the plant cell is from a tobacco plant. Even more preferably, the tobacco plant is Nicotiana benthamiana. Most preferably, the N. benthamiana is a glycosylation mutant lacking plant-specific N-glycan residues.

In a third aspect of the invention there is provided for a plant comprising or consisting of the plant cell as described herein or a plant that has been modified by the methods described herein.

### BRIEF DESCRIPTION OF THE FIGURES

Non-limiting embodiments of the invention will now be described by way of example only and with reference to the following figures:

FIG. 1: Purification and analysis of putative recombinant HIV Envelope gp140 trimers. A) Overlayed Superdex200 elution profiles of plant-produced HIV Env gp140 (Plant) and the equivalent protein produced in mammalian cells (HEK293). B) Coomassie-stained BN-PAGE of purified HIV Env gp140 from mammalian cells. C) Coomassie-stained BN-PAGE of purified HIV Env gp140 produced in Nicotiana benthamiana.

FIG. 2: Design of a soluble Marburg glycoprotein antigen (GPΔTM) for expression in plants and mammalian cells. The native signal peptide (SP) was substituted for the tissue plasminogen activator leader (TPA) sequence and the murine monoclonal leader peptide heavy chain (LPH) to facilitate expression in mammalian cells and plants, respectively. The native furin cleavage site (RRKR) was replaced with a flexible leader sequence comprising of (GGGGS)₂ to enable the protein to assume it's native confirmation in the absence of furin processing which does not naturally occur in plants. The antigen was also truncated prematurely to remove the transmembrane and cytoplasmic domains of the native protein. The location of the mucin-like domain and the GP1 and GP2 subunits are also indicated. Ecto=ectodomain, TM=transmembrane domain, Cyt=cytoplasmic domain.

FIG. 3: Gel filtration and BN-PAGE analysis of putative recombinant MARV GPΔTM trimers. A) Overlayed Superdex200 elution profiles of plant-produced MARV GPΔTM (Plant) and the equivalent protein produced in mammalian cells (HEK293). B) Coomassie-stained BN-PAGE of purified MARV GPΔTM from mammalian cells. C) Coomassie-stained BN-PAGE of purified MARV GPΔTM produced in Nicotiana benthamiana.

FIG. 4: Comparative site-specific glycosylation of recombinant HIV Env gp140 produced in plants compared to mammalian cells as determined by liquid chromatography-mass spectrometry. The differences in glycosylation are represented as the percentage point change in each glycan species when produced in plants compared to mammalian cells. Therefore, positive and negative values indicate a relative increase or decrease in a particular glycoforms when produced in plants compared to mammalian cells. The various glycan species detected are indicated in the key below the image.

FIG. 5: Comparative site-specific glycosylation of recombinant HIV Env gp140 produced in plants compared to mammalian cells as determined by liquid chromatography-mass spectrometry. The global composition of glycans are indicated for the plant (WT) and mammalian cell-produced proteins (HEK293).

FIG. 6: Comparative site-specific glycosylation of recombinant MARV GPΔTM produced in plants compared to mammalian cells as determined by liquid chromatography-mass spectrometry. The differences in glycosylation are represented as the percentage point (p.p) change in each glycan species when produced in plants compared to mammalian cells. Therefore, positive and negative values indicate a relative increase or decrease in a particular glycoforms when produced in plants compared to mammalian cells.

FIG. 7: Comparative site-specific glycosylation of recombinant MARV GPΔTM produced in plants compared to mammalian cells as determined by liquid chromatography-mass spectrometry. The global composition of glycans are indicated for the plant (WT) and mammalian cell-produced proteins (HEK293).

FIG. 8: Site-specific glycosylation of plant-produced EBV gp350ΔTM.

FIG. 9: Site-specific glycosylation of plant-produced CAP256 SU SOSIP.664.

FIG. 10: Western blotting to confirm the impact of integrated host and glyco-engineering on the production of HIV Env g140. All experimental samples were produced in N. benthamiana ΔXF plants by co-expression of human CRT to support folding. The experimental samples were produced by co-expression of LmSTT3D (CRT/LmSTT3D) and co-expression of both LmSTT3D and HEXO3RNAi (Glyco-opt.).

FIG. 11: Overlayed Superdex 200 elution profiles comparing trimer formation and resolution of recombinant HIV Env gp140 produced in HEK293 cells (HEK293), wildtype Nicotiana benthamiana (WT) following the co-expression of calreticulin and Nicotiana benthamiana ΔXF following the co-expression of host and glyco-engineering expression constructs (Glyco-opt.). The major elution peaks corresponding to aggregates (1) and trimers (2) are indicated.

FIG. 12: Coomassie-stained BN-PAGE gel of individual fractions of glyco-optimized Env gp140 following resolution on a Superdex 200 column. The numbers above each well correspond to a fraction derived from gel filtration (Superdex 200). Fractions 38-42 were pooled as trimers for subsequent studies. MW=molecular weight marker.

FIG. 13: Site-specific glycosylation of plant-produced glyco-optimized HIV Env gp140 compared to the equivalent protein produced in wildtype plants by co-expression of calreticulin. The differences in glycosylation are represented as the percentage point (p.p) change in each glycan species when produced in plants compared to mammalian cells. Therefore, positive and negative values indicate a relative increase or decrease in a particular glycoforms when produced in plants compared to mammalian cells.

FIG. 14: Site-specific glycosylation of glyco-optimized HIV Env gp140 produced in plants compared to the equivalent protein produced in mammalian cells. The differences in glycosylation are represented as the percentage point (p.p) change in each glycan species when produced in plants compared to mammalian cells. Therefore, positive and negative values indicate a relative increase or decrease in a particular glycoforms when produced in plants compared to mammalian cells.

FIG. 15: Summarized analysis of relative proportion of different glycoforms observed on recombinant plant-produced and mammalian cell-derived HIV Env gp140.

FIG. 16: Amino acid sequence of the human calreticulin protein (SEQ ID NO:2).

FIG. 17: Amino acid sequence of the human calnexin protein (SEQ ID NO:4).

FIG. 18: Amino acid sequence of the Leishmania major LmSTT3D protein (SEQ ID NO:6).

FIG. 19: Nucleic acid sequence of the sense strand of the HEXO3RNAi (SEQ ID NO:7).

FIG. 20: Nucleic acid sequence of the antisense strand of the HEXO3RNAi (SEQ ID NO:8).

FIG. 21: Site-specific glycan analysis of SARS-CoV-2 SΔTM produced in wild type N. benthamiana.

FIG. 22: Implementation of integrated host and glyco-engineering (NXS/T Generation™) to improve SARS-CoV-2 SΔTM production in plants. A) Overlayed normalized size exclusion chromatography profiles of SARS-CoV-2 SΔTM when produced in N. benthamiana wild type by co-expression of CRT (WT) or produced by integrated host and glyco-engineering in N. benthamiana ΔXF (Glyco-opt). B) Coomassie-stained BN-PAGE of pooled fractions from the “WT” SΔTM in A. C) Coomassie-stained BN-PAGE of pooled fractions from the “glyco-opt” in A. MW=molecular weight marker.

FIG. 23: Site-specific glycan analysis of “glyco-optimized” SARS-CoV-2 SΔTM.

FIG. 24: Comparison of the site-specific glycan occupancy of “glyco-optimized” and “WT” SARS-CoV-2 SΔTM. The data is presented as the percentage point change in occupation at each glycosylation sequon when the two variants of the protein are compared. Accordingly, positive value indicates an elevation in glycan occupancy in the “glyco-optimized” protein compared to the “WT protein”. Conversely, a negative value indicates decreased glycan occupancy in the “glyco-optimized” protein compared to the “WT”. * Indicates sites that were excluded from the analysis.

FIG. 25: Western blotting of crude homogenate to detect expression of a stabilized SARS-CoV-2 spike mimetic in plants. The recombinant protein was detected with polyclonal mouse anti-his tag antibody. The protein band of interest is indicated by the *. (S6ProΔTM=expression of the spike glycoprotein in the absence of accessory proteins, Protein Origami™=co-expression of the spike with human CRT in wild type N. benthamiana. NXS/T Generation™=Integration of spike co-expression with human CRT and glyco-engineering approaches that constitute the integrated host and glyco-engineering platform collectively referred to as NXS/T Generation™).

FIG. 26: Negative stain electron microscopy of purified SARS-CoV-2 spike trimers. A) Unprocessed image comprising of size exclusion chromatography-purified spike trimer mimetics. Scale bar=50 nm. B) 2D class averages and 3D reconstruction derived from A. scale bar=5 nm.

FIG. 27: Site-specific glycan analysis of SARS-CoV-2 prefusion trimers produced in N. benthamiana by integrated host and glyco-engineering (NXS/T Generation™.

FIG. 28: Negative stain electron microscopy of HEK 293-F cell-produced SARS-CoV-2 S6ProΔTM. A) Unprocessed image comprising of size exclusion chromatography-purified spike trimer mimetics. B) 2D class derived from A.

FIG. 29: Site-specific glycan analysis of SARS-CoV-2 S6ProΔTM produced in HEK293-F cells.

FIG. 30: Comparison of the site-specific glycan occupancy of “glyco-optimized” and HEK293-F-produced SARS-CoV-2 SΔTM. The data is presented as the percentage point change in occupation at each glycosylation sequon when the protein is compared between expression systems. Accordingly, positive value indicates an elevation in glycan occupancy in the “glyco-optimized” protein compared to the mammalian cell-produced. Conversely, a negative value indicates decreased glycan occupancy in the “glyco-optimized” protein compared to mammalian protein. *Indicates sites that were not determined and could not be included in the analysis.

FIG. 31: Western blotting of crude homogenate to detect expression of A) EBOV GPΔTM and B) NiV FΔTM. The recombinant proteins were detected using polyclonal mouse anti-his tag antibody which recognized the polyhistidine C-terminal tags on each antigen. The protein bands of interest are indicated by the *. (GPΔTM/FΔTM only=expression of the spike glycoprotein in the absence of accessory proteins, Protein Origami™=co-expression of the glycoprotein with human CRT in wild type N. benthamiana. NXS/T Generation™=Integration of glycoprotein co-expression with human CRT and glyco-engineering approaches).

FIG. 32: Western blotting of crude homogenate to detect expression of LUVJ GP-CΔTM following implementation of Protein Origami™ and NXS/T Generation™ approaches. A) Expression of LUJV GP-CΔTM alone (GP-CΔTM) or with the chaperone CNX or CRT. A negative control was included where the chaperone CRT was expressed alone (−ve). The samples were harvest 3 days (D3) and 5 days (D5) post agroinfiltration for analysis. B) Expression of LUJV GP-CΔTM using protein Origami™ and NXS/T Generation™ technologies. A negative control was included where the chaperone CRT was expressed alone (−ve). A positive control comprising of plant lysate containing the protein of interest was also included (+ve). Samples were harvested 3 days (D3) and 5 days (D5) post agroinfiltration. Protein Origami™ indicates the co-expression of the protein with human CRT in wild type N. benthamiana whereas NXS/T Generation™ refers to Integration of GP-CΔTM co-expression with human CRT and glyco-engineering approaches. In both A and B the recombinant protein was detected by its C-terminal tag using polyclonal mouse anti-his tag antibody. The approximate size of the protein bands of interest are indicated by the * alongside the images.

### SEQUENCE LISTING

The nucleic acid and amino acid sequences listed in the accompanying sequence listing are shown using standard letter abbreviations for nucleotide bases, and the standard three letter abbreviations for amino acids. It will be understood by those of skill in the art that only one strand of each nucleic acid sequence is shown, but that the complementary strand is included by any reference to the displayed strand. In the accompanying sequence listing:

SEQ ID NO:1 is a nucleic acid sequence of the human calreticulin protein.

SEQ ID NO:2 is an amino acid sequence of the human calreticulin protein.

SEQ ID NO:3 is a nucleic acid sequence of the human calnexin protein.

SEQ ID NO:4 is an amino acid sequence of the human calnexin protein.

SEQ ID NO:5 is a nucleic acid sequence of the Leishmania major LmSTT3D protein.

SEQ ID NO:6 is an amino acid sequence of the Leishmania major LmSTT3D protein.

SEQ ID NO:7 is a nucleic acid sequence of the sense strand of the HEXO3RNAi.

SEQ ID NO:8 is a nucleic acid sequence of the antisense strand of the HEXO3RNAi.

SEQ ID NO:9 is a nucleic acid sequence of the HIV Envelope gp140 for expression in mammalian cells.

SEQ ID NO:10 is an amino acid sequence of the HIV Envelope gp140 for expression in mammalian cells.

SEQ ID NO:11 is a nucleic acid sequence of the HIV Envelope gp140 for expression in plants.

SEQ ID NO:12 is an amino acid sequence of the HIV Envelope gp140 for expression in plants.

SEQ ID NO:13 is a nucleic acid sequence of the recombinant Marburg viral glycoprotein for expression in mammalian cells.

SEQ ID NO:14 is an amino acid sequence of the recombinant Marburg viral glycoprotein for expression in mammalian cells.

SEQ ID NO:15 is a nucleic acid sequence of the recombinant Marburg viral glycoprotein for expression in plants.

SEQ ID NO:16 is an amino acid sequence of the recombinant Marburg viral glycoprotein for expression in plants.

SEQ ID NO:17 is a nucleic acid sequence of the tissue plasminogen activator (TPA) leader sequence for the modified HIV envelope gp140 protein.

SEQ ID NO:18 is a nucleic acid sequence of the tissue plasminogen activator (TPA) leader sequence for the MARV GPΔTM antigen.

SEQ ID NO:19 is a nucleic acid sequence of the tissue plasminogen activator (TPA) leader sequence for the cleaved SOSIP.664.

SEQ ID NO:20 is an amino acid sequence of the tissue plasminogen activator (TPA) leader sequence.

SEQ ID NO:21 is a nucleic acid sequence of the murine monoclonal leader peptide heavy chain (LPH) for the modified HIV env gp140 polypeptide.

SEQ ID NO:22 is a nucleic acid sequence of the murine monoclonal leader peptide heavy chain (LPH) for the MARV GPΔTM antigen.

SEQ ID NO:23 is a nucleic acid sequence of the murine monoclonal leader peptide heavy chain (LPH) for the Epstein-Barr virus gp350 ATM.

SEQ ID NO:24 is an amino acid sequence of the murine monoclonal leader peptide heavy chain (LPH).

SEQ ID NO:25 is an amino acid sequence of the native furin cleavage site for the modified HIV env gp140 polypeptide.

SEQ ID NO:26 is an amino acid sequence of the native furin cleavage site for the MARV GPΔTM antigen.

SEQ ID NO:27 is a nucleic acid sequence of the flexible linker sequence for the modified HIV env gp140 polypeptide for expression in plant cells.

SEQ ID NO:28 is a nucleic acid sequence of the flexible linker sequence for the modified HIV env gp140 polypeptide for expression in mammalian cells.

SEQ ID NO:29 is a nucleic acid sequence of the flexible linker sequence for the MARV GPΔTM antigen for expression in plant cells.

SEQ ID NO:30 is a nucleic acid sequence of the flexible linker sequence for the MARV GPΔTM antigen for expression in mammalian cells.

SEQ ID NO:31 is an amino acid sequence of the flexible linker sequence.

SEQ ID NO:32 is a nucleic acid sequence of the Epstein-Barr virus (EBV) gp350 ATM.

SEQ ID NO:33 is an amino acid sequence EBV gp350 ATM.

SEQ ID NO:34 is a nucleic acid sequence of a cleaved SOSIP.664.

SEQ ID NO:35 is an amino acid sequence of a cleaved SOSIP.664.

SEQ ID NO:36 is a nucleic acid sequence encoding the SARS-CoV-2 SΔTM polypeptide.

SEQ ID NO:37 is an amino acid sequence of the SARS-CoV-2 SΔTM polypeptide.

SEQ ID NO:38 is a nucleic acid sequence encoding the SARS-CoV-2 S6ProΔTM polypeptide.

SEQ ID NO:39 is an amino acid sequence of the SARS-CoV-2 S6ProΔTM polypeptide.

SEQ ID NO:40 is a nucleic acid sequence encoding the Ebola virus GPΔTM polypeptide.

SEQ ID NO:41 is an amino acid sequence of the Ebola virus GPΔTM polypeptide.

SEQ ID NO:42 is a nucleic acid sequence encoding the Nipah virus FΔTM polypeptide.

SEQ ID NO:43 is an amino acid sequence of the Nipah virus FΔTM polypeptide.

SEQ ID NO:44 is a nucleic acid sequence encoding the Lujo virus GP-CΔTM polypeptide.

SEQ ID NO:45 is an amino acid sequence of the Lujo virus GP-CΔTM polypeptide.

### DETAILED DESCRIPTION OF THE INVENTION

The present invention will now be described more fully hereinafter with reference to the accompanying drawings, in which some, but not all embodiments of the invention are shown.

The invention as described should not be limited to the specific embodiments disclosed and modifications and other embodiments are intended to be included within the scope of the invention. Although specific terms are employed herein, they are used in a generic and descriptive sense only and not for purposes of limitation.

As used throughout this specification and in the claims which follow, the singular forms “a”, “an” and “the” include the plural form, unless the context clearly indicates otherwise.

The terminology and phraseology used herein is for the purpose of description and should not be regarded as limiting. The use of the terms “comprising”, “containing”, “having” and “including” and variations thereof used herein, are meant to encompass the items listed thereafter and equivalents thereof as well as additional items.

Prior to this work the bottlenecks in plants that precluded high-level production of well-folded and authentically glycosylated proteins were poorly understood. Previously, it was demonstrated that the endogenous chaperone machinery imposed a bottleneck for the efficient folding of complex glycoproteins and that the co-expression of human chaperones was necessary to support high level expression.

The inventors provide data that demonstrates that the co-expression of chaperones alone is not sufficient to produce well-folded glycoproteins in plants and that additional constraints need to be addressed to recapitulate their native structures. Specifically, the impact of the host plant glycosylation on viral glycoprotein production was poorly understood and it was not appreciated that under glycosylation and paucimannosidic/truncated glycan formation precluded the production of appropriately glycosylated and well-folded glycoproteins in the system. The under glycosylation reported here is the most extensive under glycosylation observed for a plant-produced protein to date and accounts for the extensive aggregation observed. The presence of paucimannosidic/truncated glycans is also potentially problematic as these glycans are not present in healthy human tissues and are not naturally present on viral glycoproteins from mammalian cells.

Additionally, the inventors have also determined the prevalence of plant-specific glycans in the context of plant-produced viral glycoproteins and have identified a “glycosylation signature” for heavily glycosylated viral glycoproteins trafficking to the plasma membrane. These glycans are potentially immunogenic and concerns have been raised regarding their presence following administration in humans, particularly in the context of heavily glycosylated vaccines or therapeutics or in the case where repeated administration was necessary. The inventors have therefore integrated chaperone co-expression with approaches to modify glycosylation with the intention of improving the production of recombinant HIV Env gp140 and developing a broadly applicable approach to support production of complex glycoproteins, as exemplified with several model proteins described herein. The impact of combining these approaches is not obvious as the intrinsic limitations for the molecular farming of complex glycoproteins have not been adequately determined. Not only do these approaches enable the production of well-folded and heavily glycosylated glycoproteins in plants but addressing limitations in the glycosylation machinery resulted in improved folding (decreased aggregation) and oligomerisation.

The present invention thus allows for the production of heterologous polypeptides of interest to be produced in plant cells which allow for increased expression of the heterologous polypeptide of interest, increased glycosylation efficiency of the heterologous polypeptide of interest, a reduction in plant specific modifications of the heterologous polypeptide of interest, a reduction in aggregation of the heterologous polypeptide of interest; and/or correct folding and oligomerisation of the heterologous polypeptide of interest. By improving the glycosylation and glycosylation-directed folding of the heterologous polypeptide of interest the invention enables reduction of undesired glycoforms, promotes the correct folding of the polypeptide of interest and prevents aggregation of the polypeptide of interest. Additionally, the correct folding of the polypeptide of interest results in less aggregation and improved formation of desired oligomers, such as trimers thereby enabling recapitulation of the native structure of the glycoprotein.

These approaches have far-reaching ramifications for the molecular farming of complex glycoprotein-based pharmaceuticals in plants. The integration of the approaches described herein now enables the production of proteins which could not previously be produced at sufficient levels, or in the appropriate conformations in plants. This technology results in the production of recombinant glycoproteins which lack undesired plant-specific glycans and contain similar glycan occupancy to mammalian proteins. This work therefore enables the production of virus-like particles and synthetic nanoparticles vaccines which display well-folded and appropriately glycosylated viral glycoproteins for the first time. These approaches are similarly applicable to therapeutic glycoproteins, such as antibodies, and the production of cancer antigens, and recombinant antigens which can be applied as therapeutics, used as research or serology reagents and applied in diagnostic tests. The invention will further enable the generation of glycoproteins with tailored glycan profiles by extension of the glycan structure to impart mammalian-type fucosylation, galactosylation and sialylation. Ultimately, this technology enables both the production of these proteins and their modification to improve their immunogenicity or potency.

As used herein the terms “protein,” “peptide” or “polypeptide” are used interchangeably and refer to any chain of two or more amino acids, including naturally occurring or non-naturally occurring amino acids or amino acid analogues, irrespective of post-translational modification (e.g., glycosylation or phosphorylation). The amino acids are thus in a polymeric form of any length, linked together by peptide bonds.

The term “heterologous polypeptide of interest” or “polypeptide of interest” as used herein refers to any polypeptide that does not occur naturally in a plant. A heterologous polypeptide of interest may thus include protozoal, bacterial, viral, fungal or animal proteins. The heterologous polypeptide of interest is intended for expression in a plant cell or plant tissue using the methods of the present invention. Non-limiting examples of heterologous polypeptides of interest may include, pharmacological polypeptides (e.g., for medical uses, for cell- and tissue culture) or industrial polypeptides (e.g. enzymes, growth factors) that can be produced according to the methods present invention. The heterologous polypeptides of interest may be useful as vaccines or for use in vaccines, as well as in other reagents or diagnostics.

As used herein the term “plant cell which is transformed” refers to a plant or plant cell which has either been stably transformed in order to express a heterologous polypeptide or which has been infiltrated with at least one expression vector which transiently expresses a heterologous polypeptide in the plant or plant cell.

The terms “nucleic acid”, “nucleic acid molecule” and “polynucleotide” are used herein interchangeably and encompass both ribonucleotides (RNA) and deoxyribonucleotides (DNA), including cDNA, genomic DNA, and synthetic DNA. The nucleic acid may be double-stranded or single-stranded. Where the nucleic acid is single-stranded, the nucleic acid may be the sense strand or the antisense strand. A nucleic acid molecule may be any chain of two or more covalently bonded nucleotides, including naturally occurring or non-naturally occurring nucleotides, or nucleotide analogs or derivatives. The term “DNA” refers to a sequence of two or more covalently bonded, naturally occurring or modified deoxyribonucleotides.

The term “isolated”, is used herein and means having been removed from its natural environment.

The term “purified”, relates to the isolation of a molecule or compound in a form that is substantially free of contamination or contaminants. Contaminants are normally associated with the molecule or compound in a natural environment, purified thus means having an increase in purity as a result of being separated from the other components of an original composition. The term “purified nucleic acid” describes a nucleic acid sequence that has been separated from other compounds including, but not limited to polypeptides, lipids and carbohydrates which it is ordinarily associated with in its natural state.

The term “complementary” refers to two nucleic acids molecules which are capable of forming Watson-Crick base pairs to produce a region of double-strandedness between the two nucleic acid molecules. It will be appreciated by those of skill in the art that each nucleotide in a nucleic acid molecule need not form a matched Watson-Crick base pair with a nucleotide in an opposing complementary strand to form a duplex. One nucleic acid molecule is thus “complementary” to a second nucleic acid molecule if it hybridizes, under conditions of high stringency, with the second nucleic acid molecule. A nucleic acid molecule according to the invention includes both complementary molecules.

As used herein a “substantially identical” sequence is an amino acid or nucleotide sequence that differs from a reference sequence only by one or more conservative substitutions, or by one or more non-conservative substitutions, deletions, or insertions located at positions of the sequence that do not destroy or substantially reduce the antigenicity of one or more of the expressed polypeptides or of the polypeptides encoded by the nucleic acid molecules. Alignment for purposes of determining percent sequence identity can be achieved in various ways that are within the knowledge of those with skill in the art. These include using, for instance, computer software such as ALIGN, Megalign (DNASTAR), CLUSTALW or BLAST software. Those skilled in the art can readily determine appropriate parameters for measuring alignment, including any algorithms needed to achieve maximal alignment over the full length of the sequences being compared. In one embodiment of the invention there is provided for a polypeptide or polynucleotide sequence that has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or 100% sequence identity to the sequences described herein.

Alternatively, or additionally, two nucleic acid sequences may be “substantially identical” if they hybridize under high stringency conditions. The “stringency” of a hybridisation reaction is readily determinable by one of ordinary skill in the art, and generally is an empirical calculation which depends upon probe length, washing temperature, and salt concentration. In general, longer probes required higher temperatures for proper annealing, while shorter probes require lower temperatures. Hybridisation generally depends on the ability of denatured DNA to re-anneal when complementary strands are present in an environment below their melting temperature. A typical example of such “stringent” hybridisation conditions would be hybridisation carried out for 18 hours at 65° C. with gentle shaking, a first wash for 12 min at 65° C. in Wash Buffer A (0.5% SDS; 2×SSC), and a second wash for 10 min at 65° C. in Wash Buffer B (0.1% SDS; 0.5% SSC).

Those skilled in the art will appreciate that polypeptides, peptides or peptide analogues can be synthesised using standard chemical techniques, for instance, by automated synthesis using solution or solid phase synthesis methodology. Automated peptide synthesisers are commercially available and use techniques known in the art. Polypeptides, peptides and peptide analogues can also be prepared from their corresponding nucleic acid molecules using recombinant DNA technology.

As used herein, the term “gene” refers to a nucleic acid that encodes a functional product, for instance a RNA, polypeptide or protein. A gene may include regulatory sequences upstream or downstream of the sequence encoding the functional product.

As used herein, the term “coding sequence” refers to a nucleic acid sequence that encodes a specific amino acid sequence. On the other hand a “regulatory sequence” refers to a nucleotide sequence located either upstream, downstream or within a coding sequence. Generally regulatory sequences influence the transcription, RNA processing or stability, or translation of an associated coding sequence. Regulatory sequences include but are not limited to: effector binding sites, enhancers, introns, polyadenylation recognition sequences, promoters, RNA processing sites, stem-loop structures, translation leader sequences and the like.

The term “RNA interference” or “RNAi” refers to a process in which a double-stranded RNA molecule changes the expression of a nucleic acid sequence with which the double-stranded or short hairpin RNA molecule shares substantial or total homology. The term “RNAi agent” refers to an RNA sequence that elicits RNAi and the term “ddRNAi agent” refers to an RNAi agent that is transcribed from a vector. The terms “short hairpin RNA” or “shRNA” refer to an RNA structure having a duplex region and a loop region. In mammals, RNA interference, or RNAi, is mediated by 15- to 49-nucleotide long, double-stranded RNA molecules referred to as small interfering RNAs (RNAi agents). RNAi agents can be synthesized chemically or enzymatically outside of cells and subsequently delivered to cells or can be expressed in vivo by an appropriate vector.

The term “chaperone” refers to polypeptides which facilitate protein folding by non-enzymatic means, in that they do not catalyse the chemical modification of any structures in folding polypeptides. Chaperones potentiate the correct folding of polypeptides by facilitating correct structural alignment thereof. Molecular chaperones are well known in the art and several families thereof have previously been characterised. It is envisioned that for the purposes of the present invention any molecular chaperone protein will be suitable for use, including chaperone proteins derived from a host organism best suited to the expression of a heterologous protein of interest. In one embodiment the chaperone protein includes cytoplasmic chaperones, cytosolic chaperones or endoplasmic reticulum chaperones from other plants, animals, insects, humans, yeast or fungi. In an alternative embodiment the chaperone protein is a mammalian chaperone protein, preferably a human chaperone protein, selected from the group consisting of general chaperones, lectin chaperones, and non-classical chaperones. The term chaperone includes molecular chaperones selected from the following non-exhaustive group: calnexin, calreticulin, GRP78/BiP, GRP94, GRP170, HSP47, ERp29, Protein disulfide isomerase (PDI), peptidyl prolyl cis-trans-isomerase (PPI), and ERp57. Further, the chaperones may be expressed in combinations or co-expressed with oligosaccharyltransferases, and other glycan-modifying enzymes to improve the glycosylation. For example Leishmania major LmSTT3D may be co-expressed with calreticulin, to improve the glycan occupancy of the recombinant HIV-1 gp140 Env proteins or other glycoproteins. Similarly, other heterologous oligosaccharyltransferase enzymes may also be used.

As used herein, the term “glycoprotein” refers to a glycoprotein that would normally be produced in a mammalian cell, including viral glycoproteins or viruses having a mammalian host, and antibodies.

In some embodiments, the genes used in the method of the invention may be operably linked to other sequences. By “operably linked” is meant that the nucleic acid molecules encoding the recombinant polypeptides of the invention and regulatory sequences are connected in such a way as to permit expression of the proteins when the appropriate molecules are bound to the regulatory sequences. Such operably linked sequences may be contained in vectors or expression constructs which can be transformed or transfected into host cells for expression. It will be appreciated that any vector or vectors can be used for the purposes of expressing the recombinant antigenic polypeptides of the invention.

The term “promoter” refers to a DNA sequence that is capable of controlling the expression of a nucleic acid coding sequence or functional RNA. A promoter may be based entirely on a native gene or it may be comprised of different elements from different promoters found in nature. Different promoters are capable of directing the expression of a gene in different cell types, or at different stages of development, or in response to different environmental or physiological conditions. A “constitutive promoter” is a promoter that direct the expression of a gene of interest in most host cell types most of the time.

The term “recombinant” means that something has been recombined. When used with reference to a nucleic acid construct the term refers to a molecule that comprises nucleic acid sequences that are joined together or produced by means of molecular biological techniques. The term “recombinant” when used in reference to a protein or a polypeptide refers to a protein or polypeptide molecule which is expressed from a recombinant nucleic acid construct created by means of molecular biological techniques. Recombinant nucleic acid constructs may include a nucleotide sequence which is ligated to, or is manipulated to become ligated to, a nucleic acid sequence to which it is not ligated in nature, or to which it is ligated at a different location in nature. Accordingly, a recombinant nucleic acid construct indicates that the nucleic acid molecule has been manipulated using genetic engineering, i.e. by human intervention. Recombinant nucleic acid constructs may be introduced into a host cell by transformation. Such recombinant nucleic acid constructs may include sequences derived from the same host cell species or from different host cell species.

The term “vector” refers to a means by which polynucleotides or gene sequences can be introduced into a cell. There are various types of vectors known in the art including plasmids, viruses, bacteriophages and cosmids. Generally polynucleotides or gene sequences are introduced into a vector by means of a cassette. The term “cassette” refers to a polynucleotide or gene sequence that is expressed from a vector, for example, the polynucleotide or gene sequences encoding the acyl transferase polypeptides of the invention. A cassette generally comprises a gene sequence inserted into a vector, which in some embodiments, provides regulatory sequences for expressing the polynucleotide or gene sequences. In other embodiments, the vector provides the regulatory sequences for the expression of the acyl transferase polypeptides. In further embodiments, the vector provides some regulatory sequences and the nucleotide or gene sequence provides other regulatory sequences. “Regulatory sequences” include but are not limited to promoters, transcription termination sequences, enhancers, splice acceptors, donor sequences, introns, ribosome binding sequences, poly(A) addition sequences, and/or origins of replication.

The following examples are offered by way of illustration and not by way of limitation.

### Example 1

### Identification of Host Glycosylation as a Critical Bottleneck for Producing Complex Glycoproteins in Plants

Previous work in our group has demonstrated that the host chaperone machinery in plants does not support efficient production of complex glycoproteins. Accordingly, we demonstrated that the co-expression of the lectin-binding chaperones (calnexin (SEQ ID NO:4) and calreticulin (SEQ ID NO:2)) improved production of heavily glycosylated viral glycoproteins in plants. Patent applications have been filed in order to protect the underlying technology, and to enable the pipeline to be further developed for commercialization (see for instance International Publication No. WO 2018/220595 and International Publication No. WO 2021/220246). However, despite the improved yields of viral glycoproteins in plants, considerable aggregation of the recombinant proteins was observed suggesting further constraints precluding their efficient production.

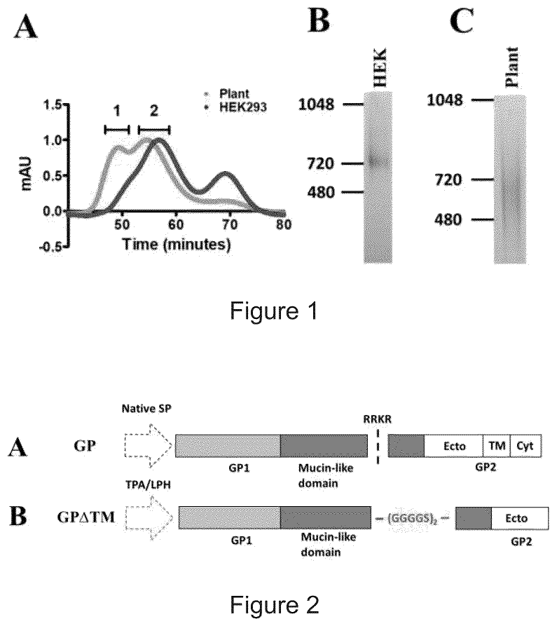

HIV Envelope gp140 (SEQ ID NO:12) as described in International Patent Publication No. WO 2018/069878, was transiently expressed in wildtype Nicotiana benthamiana by co-expression of human calreticulin (SEQ ID NO:2) and purified by Galanthus nivalis lectin affinity chromatography and gel filtration. The equivalent protein (SEQ ID NO:10) was also expressed in HEK293 cells and purified using the same approach. The Superdex200 elution profiles of both antigens were overlayed to compare their heterogeneity and efficiency of trimer formation (FIG. 1A). The elution of the plant-produced protein exhibited a pronounced shift towards the left of the profile indicating an increase in size compared to the mammalian cell-produced protein (HEK293). The plant-derived antigen exhibited 2 main peaks which comprise of aggregates (indicated as “1” in FIG. 1A) and trimers (indicated as “2” in FIG. 1A), respectively. The prominent aggregate peak is highly undesirable as protective antibody responses are believed to preferentially target the trimeric conformation of the protein. In contrast, the mammalian cell-produced protein contained only a small shoulder corresponding to aggregates, with the most abundant protein species being trimeric. This data demonstrates increased aggregation in plants following production of the recombinant glycoprotein, and that oligomerisation is inefficient in plant cells. The elution fractions comprising the putative trimer peak were pooled and concentrated. The purified trimers were then resolved by BN-PAGE and stained with BioSafe Coomassie G250 to verify their oligomeric identity (FIG. 1B and FIG. 1C). The mammalian protein (HEK) yielded a defined band of the expected size for trimeric antigens (˜720 kDa) (FIG. 1B) whereas the plant-produced protein yielded a diffuse smear that was poorly resolved by BN-PAGE (FIG. 1C).

In order to investigate if this effect was specific to the HIV Envelope glycoprotein or a reflection of the plant production system, a recombinant Marburg viral glycoprotein (SEQ ID NO:16) was similarly produced based on Lake Victoria isolate (strain Musoke-80, UniProt accession #P35253). The gene was designed as a soluble derivative of the full-length glycoprotein (FIG. 2) to support high level expression in both plants (SEQ ID NO:15) and mammalian cells (SEQ ID NO:13).

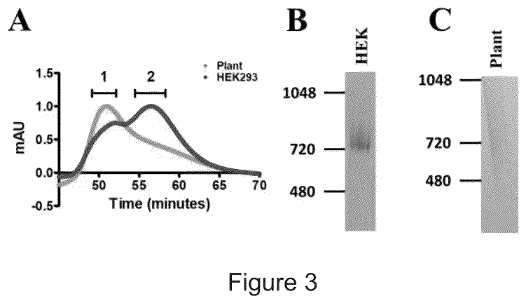

The protein (SEQ ID NO:16) was transiently expressed in N. benthamiana with human calreticulin (SEQ ID NO:2) by Agroinfiltration and purified as described for HIV Env gp140. Gel filtration using a Superdex 200 resin yielded a similar result to what was observed for HIV Env gp140 with an obvious shift of the plant-produced protein towards the left of the profile (FIG. 3A). The mammalian cell-produced antigen yielded a predominant trimer peak with some aggregates observed, whereas the plant-produced protein yielded predominantly aggregates and a diffuse shoulder containing the trimer fraction (FIG. 3A). This result was mirrored by Coomassie stained BN-PAGE gels of the pooled and concentrated trimers (FIG. 3A and FIG. 3B). The mammalian cell-produced protein (SEQ ID NO:14) yielded a defined band of ˜720 kDa (FIG. 3B) whereas the plant-produced protein yielded a diffuse smear (FIG. 3C) that was poorly resolved. This data confirms that increased aggregation of plant-produced viral glycoproteins is not unique to the HIV Env glycoprotein but is rather a reflection of the plant expression system. Appropriate oligomerisation is similarly impaired in plants.

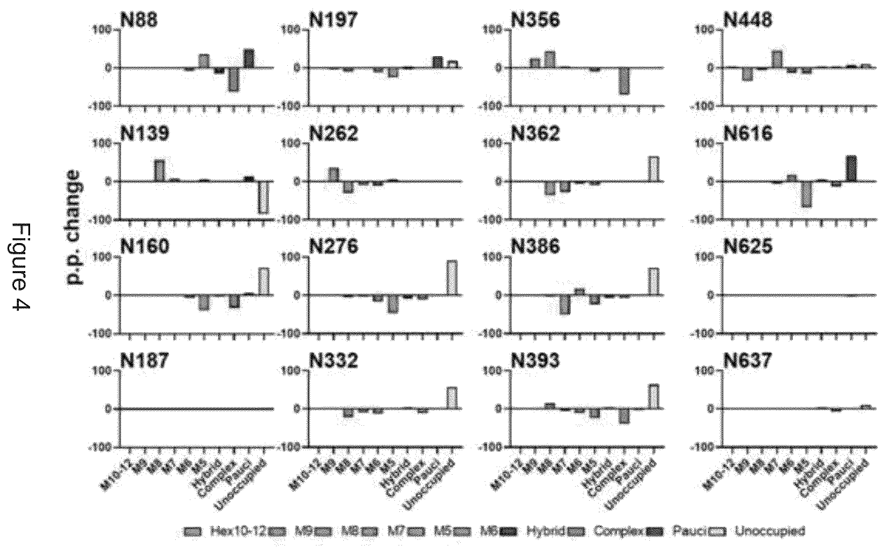

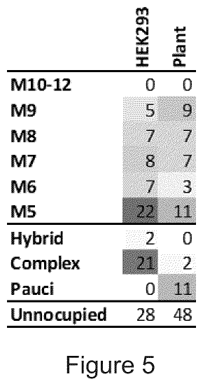

This data suggested that the plant expression platform did not support the efficient production of complex glycoproteins and suggested that additional constraints beyond the chaperone machinery may prevent appropriate glycoprotein folding and oligomerisation. Given the central role of glycosylation in protein folding, the site-specific glycosylation was determined by liquid chromatography-mass spectrometry in order to establish a potential molecular basis for the inefficient trimer formation in plants. The site-specific glycan occupancy of the HIV and Marburg proteins were determined and compared to the equivalent mammalian cell-produced antigens (FIGS. 4 & 5). This data revealed extensive under occupancy of putative N-glycosylation sites in plants compared to mammalian cells. This is the most extensive under glycosylation observed in a plant-produced protein and accounts for the high levels of aggregation observed in FIG. 1. It is of particular interest that the glycosylation sites N160 and N332 exhibit considerably lower levels of glycosylation than the mammalian cell-produced protein as the glycans at these sites comprise important components of epitopes targeted by broadly neutralizing antibodies. In addition, the plant-produced protein contained decreased complex glycans and elevated truncated glycans (pauci) which were lacking in the mammalian cell-produced material.

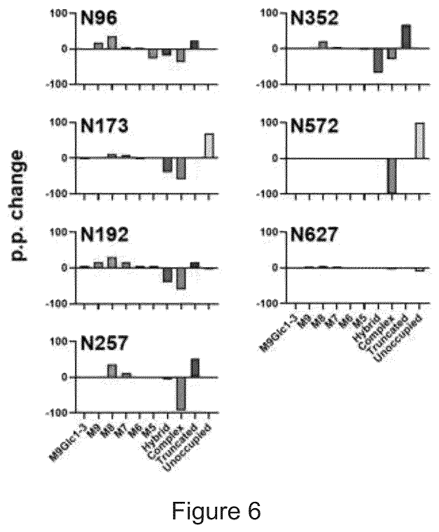

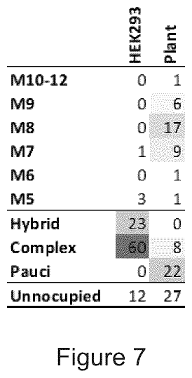

The site-specific glycosylation of the plant-produced and mammalian cell-derived MARV GPΔTM antigens were similarly compared (FIGS. 6 & 7). This analysis mirrored the observations for recombinant HIV Env gp140 revealing large amounts of under occupied sites when produced in plants compared to mammalian cells. Similarly, the plant-produced material contained decreased complex glycans and a large proportion of truncated (pauci) glycans.

In order to verify that these observations represented a glycosylation signature for heavily glycosylated glycoproteins produced in plants, the glycosylation of soluble Epstein-Barr virus (EBV) gp350 ATM (SEQ ID NO:33, FIG. 8) and a cleaved SOSIP.664 (SEQ ID NO:35, FIG. 9) from a previous study were determined. The data generated was consistent with the previous analysis and large amounts of under occupied glycan sites were observed, as well as truncated/paucimannosidic glycans and low levels of plant-specific complex glycans.

Collectively this data demonstrates a glycosylation signature for complex plant-produced glycoproteins and identifies key constraints for their production in plants. This work was facilitated by the co-expression of chaperones which were a prerequisite to enable sufficient levels of material to be produced for analysis. However, in order to produce well-folded and appropriately glycosylated viral glycoproteins in plants both chaperone-mediated folding and host glycosylation needs to be supported. This data addresses a critical knowledge gap to facilitate the development of an appropriate intervention to enable the production of these proteins in plants where they reproduce critical features of the native protein that are required for folding, oligomerisation, biological activity and immunogenicity as a vaccine. In brief, the data shows that in order to produce well-folded and appropriately glycosylated complex glycoproteins chaperone co-expression is necessary to support folding, glycan occupancy needs to be increased and the activity of endogenous hexosaminidase enzymes needs to be mitigated to prevent formation of truncated (paucimannosidic glycans).

### Example 2

Synthetic DNA encoding the genes of interest were commercially synthesized for heterologous expression. The chaperone and glycoprotein sequences were optimized to reflect the preferred human codon usage whereas the glyco-engineering cassettes were modified to reflect the preferred plant codon usage. Both the HIV Env gp140 (SEQ ID NO:11) and MARV GPΔTM (SEQ ID NO:15) coding sequence was modified by replacing the native leader sequence with the heterologous tissue plasminogen activator sequence (TPA) or murine monoclonal antibody leader peptide heavy chain (LPH) sequence for expression in mammalian cells and plants, respectively. The HIV coding sequence was further modified by including an isoleucine to proline stabilizing mutation at residue 559. In both glycoproteins the native furin cleavage site was replaced with a flexible linker peptide (GGGGS₂) (SEQ ID NO:31). The HIV gene was terminated at residue 664 whereas the MARV GPΔTM gene was truncated at residue 648 to remove the transmembrane and cytoplasmic regions.

The chaperone and glycoprotein genes were cloned into PEAQ-HT and transformed into A. tumefaciens AGL1. The LmSTT3D (SEQ ID NO:5) was cloned into p47 and HEXO3RNAi sequences (sense SEQ ID NO:7; antisense SEQ ID NO:8) were cloned into pPT2 and transformed into A. tumefaciens GV3101:pMP90. Recombinant A. tumefaciens strains were cultivated in Luria Bertani base media (12.5 g/l yeast extract, 2.5 g/l tryptone, 5 g/l NaCl, 10 mM MES [pH 5.6], with antibiotic selection (Table 1). Recombinant A. tumefaciens were stored as glycerol stocks at −80° C. and revived in 10 ml of culture medium for infiltrations. Starter cultures were systematically scaled up to 1 litre for infiltrations and the final culture inoculum was supplemented with 20 μM acetosyringone. On the day of infiltration the OD₆₀₀ of each culture was determined and the bacterial inocula were mixed and adjusted to a final OD₆₀₀ as outlined in table 1 using resuspension media (10 mM MgCl₂, 10 mM MES [pH5.], 200 μM acetosyringone. Plants were infiltrated with the bacterial suspensions at 6-8 weeks of age and then returned to the green house for incubation under controlled conditions.

TABLE 1
Summary of expression constructs, antibiotic selection and expression parameters

| Protein | vector | A. tumefaciens | OD₆₀₀ | Selection | Function | SEQ ID |
|---|---|---|---|---|---|---|
| Calreticulin | pEAQ-HT | AGL1 | 0.5 | Kan⁵⁰, carb⁵⁰ | Chaperone | SEQ ID NO: 2 |
| Calnexin | pEAQ-HT | AGL1 | 0.5 | Kan⁵⁰, carb⁵⁰ | Chaperone | SEQ ID NO: 4 |
| LmSTT3D | P47 | GV3101:pMP90 | 0.25 | Gent⁵⁰, Rif⁵⁰, Spec⁵⁰ | OST | SEQ ID NO: 6 |
| HEXO3RNAi | pPT2 | GV3101:pMP90 | 0.25 | Gent⁵⁰, Rif⁵⁰, Kan⁵⁰ | RNAi | SEQ ID NO: 7, 8 |
| Glycoprotein | pEAQ-HT | AGL1 | 0.5 | Kan⁵⁰, carb⁵⁰ | Antigen | SEQ ID NO: 16 |

Protein and sampling was performed 4-5 days post agroinfiltration. Small scale isolations were conducted to recover crude leaf lysate for western blotting by homogenizing leaf clippings in liquid nitrogen. The cell lysate was resuspended in 2 buffer volumes of phosphate or Tris-based buffer with an appropriate pH. Buffers were supplemented with Depol 40 to macerate the cell wall, EDTA-free protease inhibitors and in some cases detergents or urea to solubilize the antigen. The homogenate was incubated at 4° C. for 1 hour with shaking and then clarified at 15000G. The supernatant was retained for western blotting.

Large scale protein isolations were conducted under conditions to preserve the native protein conformation. The aerial parts of the leaf were recovered 4-5 days post-agroinfiltration and were homogenized in 2 buffer volumes of extraction buffer. Extraction buffers were Tris or phosphate-based and were supplemented with Depol 40 and EDTA-free protease inhibitor. The plant homogenate was incubated for 1 hour at 4° C. with shaking to maximize recovery of the protein. The homogenate was then filtered through Miracloth and clarified at 17000G. The clarified lysate was filtered through a 0.45 μM stericup filter and applied to a Galanthus nivalis lectin affinity column under control of a peristaltic pump. The bound protein was sequentially washed with 10 column volumes of 0.5 M NaCl and PBS, and then eluted with 1M Methyl α-D-mannopyranoside for 2 hours at 10 rpm. The eluate was concentrated to 5 ml and buffer exchanged into PBS [pH7.4] using a centrifugal column concentrator. The concentrated eluate was filtered through a 0.22 μM filter and then injected onto a Superdex 200 column which had been equilibrated with PBS, or a comparable Tris-based buffer. Individual fractions comprising the elution peaks were recovered and analyzed by resolving them on BN-PAGE gels that were stained with Coomassie. Fractions corresponding to the desired protein species were pooled and stored at −80° C. for further analysis. In some cases, the pooled size exclusion chromatography fractions were further concentrated using centrifugal column concentrators.

### Example 3

### Integrated Host and Glyco-Engineering Approaches Support the Production of a Glyco-Optimized HIV Env Gp140 Antigen

Following determination of the site-specific glycosylation of the plant-produced viral glycoproteins an integrated expression approach was conceived to support improved production of a prototype HIV Envelope gp140 glycoprotein in plants. This approach was conceived to address host constraints precluding efficient production and glycosylation of the recombinant protein:

1. Human calreticulin (SEQ ID NO:2) was co-expressed to support protein folding and improve expression yields

2. Leishmania major LmSTT3D (SEQ ID NO:6) was co-expressed to improve glycan occupancy

3. An RNA interference construct was co-expressed to suppress Hexosaminidase 3 (HEXO3RNAi) (sense SEQ ID NO:7, antisense SEQ ID NO:8) which is responsible for the formation of truncated (paucimannosidic) glycans.

4. Protein production was conducted using Nicotiana benthamiana ΔXF plants which have been modified to mitigate activities of the enzymes responsible for imparting plant-specific complex glycans.

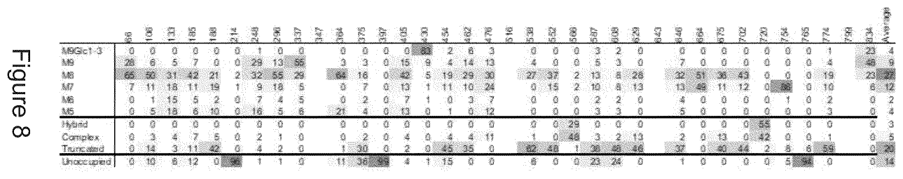

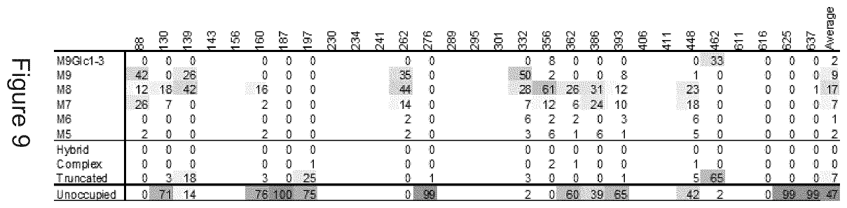

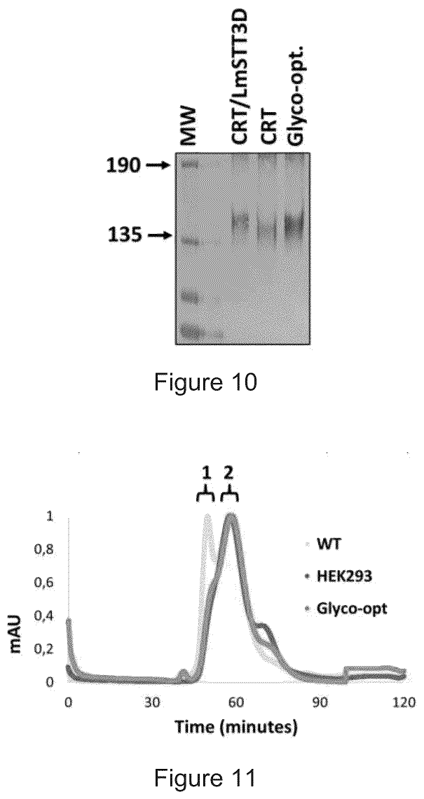

These approaches were combined with the transient expression of HIV Env gp140 (SEQ ID NO:12) and leaf material was harvested 4-5 days post agroinfiltration. This integrated approach (glyco-optimized) was compared to plants infiltrated with a) gp140 and CRT and b) gp140/CRT/LmSTT3D. Crude leaf lysate was resolved by SDS-PAGE and subjected to western blotting using polyclonal goat-anti-gp120. Both samples where LmSTT3D were co-expressed (FIG. 10) had a larger molecular weight suggesting an increase in glycan occupancy compared to the control sample (CRT). Given that each glycan is expected to add 2-3 kDa to the protein backbone, the increase in glycan occupancy must be considerable to yield a visible size increase following western blotting.

In order to further verify the impact of the integrated glyco-optimized co-expression approach, the production of the glyco-optimized gp140 antigen was scaled up. The recombinant protein was purified by sequential Galanthus nivalis lectin and size exclusion chromatography procedures. Size exclusion chromatography was performed using a Superdex 200 column and the elution profile of the glyco-optimized protein (Glyco-opt) was overlayed with the equivalent protein produced in mammalian cells (HEK293) and the protein produced in wildtype Nicotiana benthamiana by co-expression of calreticulin (CRT) (FIG. 11).

The protein produced in wildtype plants, in the absence of glyco-engineering, yielded a prominent aggregate peak which was not observed in the mammalian cell-produced sample or in the glyco-optimized sample. In contrast, both the glyco-optimized sample and the HEK293 sample yielded comparatively low levels of aggregates and the predominant peak was composed of trimers. Encouragingly, the elution profiles of the glyco-optimized protein overlaid perfectly with the HEK293 protein suggesting that they were comparable. This data demonstrates that the aggregation was due to impaired glycosylation that occurred following expression in plants. The data also demonstrates that the integrated host engineering approaches improved the glycosylation, folding and oligomerisation resulting in an antigen that was comparable to the mammalian cell-produced protein.

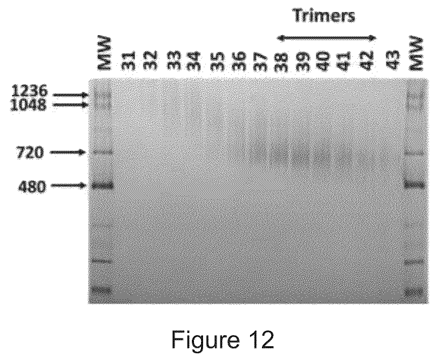

Coomassie-stained BN-PAGE gels of individual fractions of the glyco-optimized HIV Env gp140 derived from gel filtration demonstrated efficient resolution of aggregates and trimers (FIG. 12). Compared to the protein produced in FIG. 1, the purified glyco-optimized protein yielded a product of the expected size for trimeric Env gp140 and size exclusion enabled the removal of undesired aggregates and enrichment for trimeric protein.

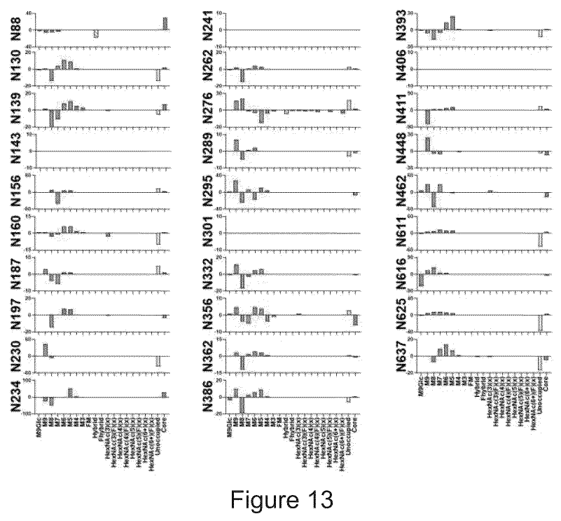

The site-specific glycosylation of the glyco-optimized protein was subsequently determined and compared to the equivalent protein produced in wildtype plants following co-expression of human calreticulin (FIG. 13). This data confirmed the successful integration of host and glycoengineering to produce a recombinant glycoprotein that had improved glycosylation and which contained negligible undesirable plant-specific modifications. The glyco-optimized protein contained decreased under occupied glycan sites (i.e the glycosylation increased) and undesirable plant-specific modifications. This data represents and incremental improvement in the glycosylation demonstrating the need to integrate both chaperone co-expression and glyco-engineering to facilitate production of complex glycoproteins in plants. Notably, the improvement in glycosylation observed was associated with a concomitant improvement in protein folding and oligomerisation.

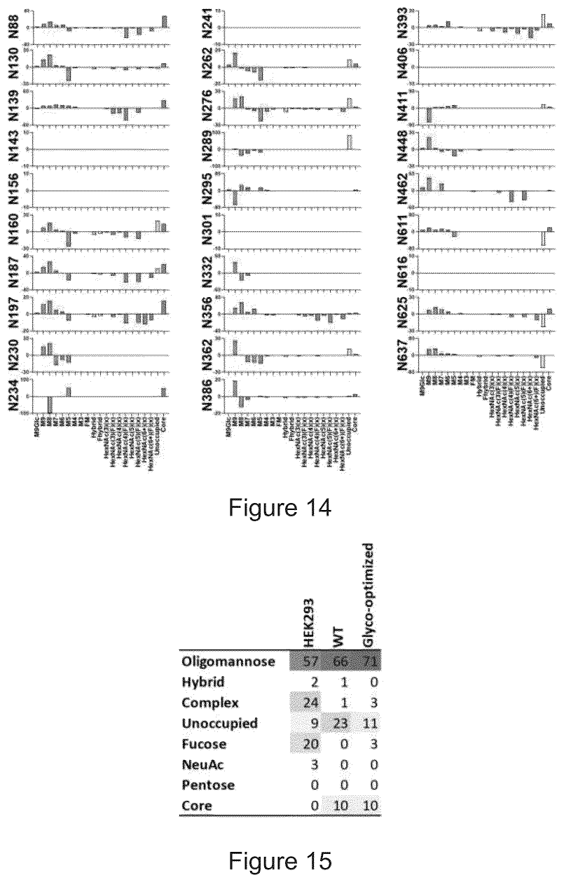

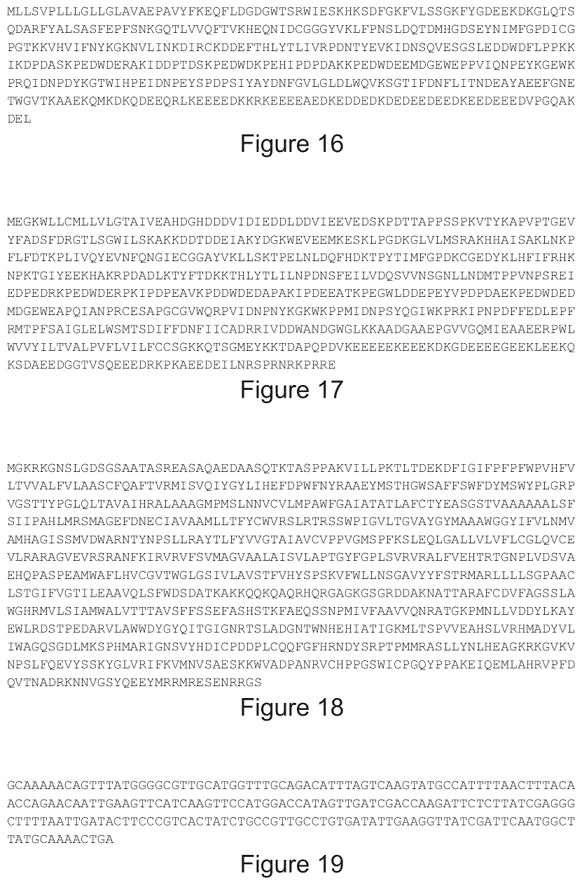

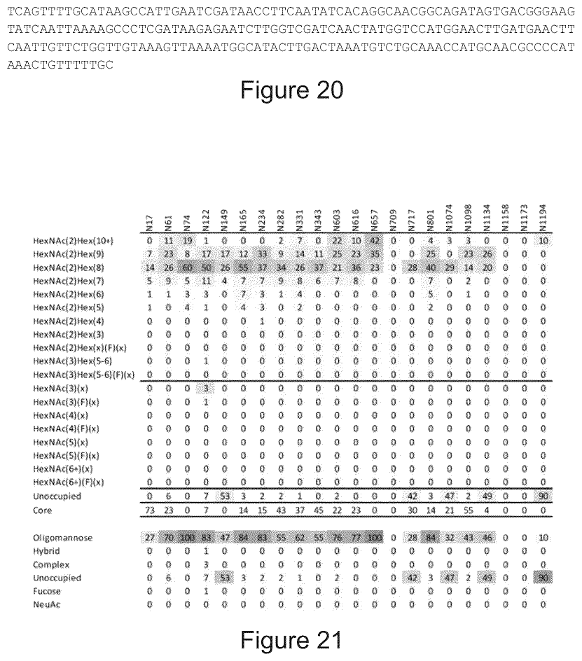

The glycosylation of the glyco-optimized protein was similarly compared to the mammalian cell-produced antigen (FIG. 14). The glycan occupancy of the 2 proteins were largely comparable, although subtle differences were observed at several sites. In some cases the plant-produced protein had increased levels of occupancy whereas at other sites the inverse was observed. Of particular interest is the observation that the glycosylation site at N332 that is targeted by neutralizing antibodies had comparable occupancy between the 2 proteins, whereas the site at N160 had increased occupancy in plants. As expected the plant-derived protein had decreased complex glycoforms due to production in N. benthamiana ΔXF plants which prevent the formation of complex glycans. Comparison of the global glycosylation of the 3 recombinant proteins (FIG. 15) highlights the considerable improvements in glycosylation achieved using the integrated approaches and suggests that this strategy now enables the production of authentic glycoproteins in plants which recapitulate the important features of the native protein including glycosylation, folding and oligomerisation.

### Example 4

### Integrated Host and Glyco-Engineering Improves Production of a SARS-CoV-2 Spike in Plants

In order to further verify that the glycosylation patterns observed reflected a common signature for plant-produced viral glycoproteins, we also determined the site-specific glycosylation of a SARS-CoV-2 spike antigen produced in N. benthamiana; as a prototype antigen for an emerging virus. SARS-CoV-2 SΔTM (SEQ ID NO:37; described in International Patent Publication No. WO 2021/220246) was produced by co-expression of human calreticulin (described in International Patent Publication No. WO 2021/220246) and then purified by Galanthus nivalis lectin affinity chromatography. Determination of the site-specific glycosylation confirmed aberrant glycosylation in plants including unoccupied potential N-linked glycosylation sites and truncated glycans at multiple sequins (FIG. 21). The predominant glycan population that was observed comprised of oligomannose-type structures with variable degrees of mannose processing.

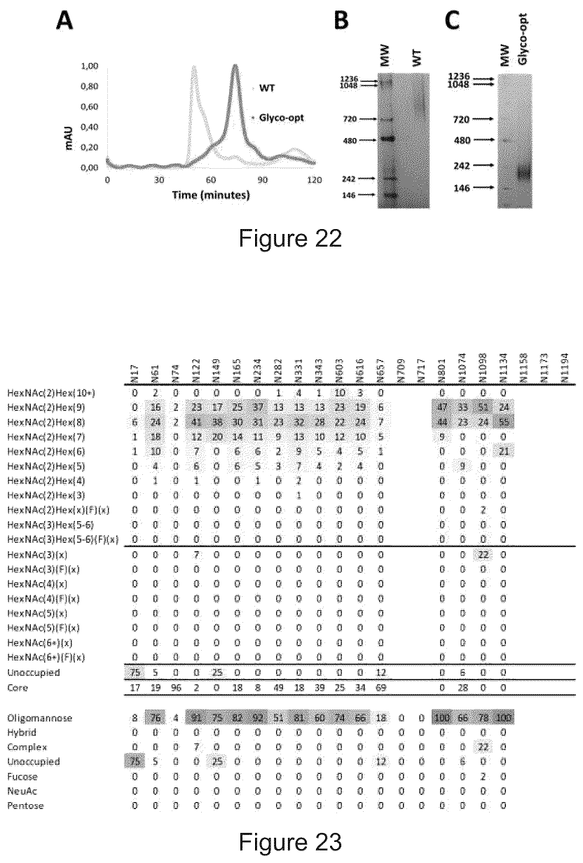

Accordingly, we applied the integrated host and glyco-engineering approach (NXS/T Generation™) described in Example 3 to improve the production of the SΔTM antigen in plants (subsequently referred to as “glyco-optimized”). This involves the co-expression of the human chaperone calreticulin, co-expression of Leishmania major LmSTT3D and RNAi-mediated suppression of endogenous HEXO3 activity. These approaches were combined using N. benthamiana ΔXF as an expression host. The protein was purified 4 days post agroinfiltration by sequential GNL-affinity chromatography and gel filtration procedures. The protein was also produced by co-expression of calreticulin only, using wild type N. benthamiana plants for comparative purposes (referred to as “WT”). The gel filtration profiles were overlayed to determine the impact of integrating host and glyco-engineering (FIG. 22A), and the proteins were resolved by BN-PAGE and then stained with Bio-Safe™ Coomassie stain (FIG. 22B and FIG. 22C). The “WT” SΔTM exhibited an overt shift to the left of the size exclusion chromatography profile, consistent with the formation of aggregated protein (FIG. 22A). In contrast, the “glyco-optimized” protein yielded a peak to the right of the profile which is consistent with a smaller product. BN-PAGE analysis of the two variants mirrored these observations. The “WT” SΔTM protein yielded a diffuse smear of the expected size for higher order protein aggregates (FIG. 22B). This also confirmed considerable heterogeneity in the purified product. The “glyco-optimized” protein yielded a defined band of ˜242 kDa when resolved by BN-PAGE (FIG. 22C). The “glyco-optimized” product demonstrated improved homogeneity and the resolution was also superior to the “WT”. In the absence of integrated host and glyco-engineering approaches, the resulting “WT” protein comprised predominantly of aggregates.

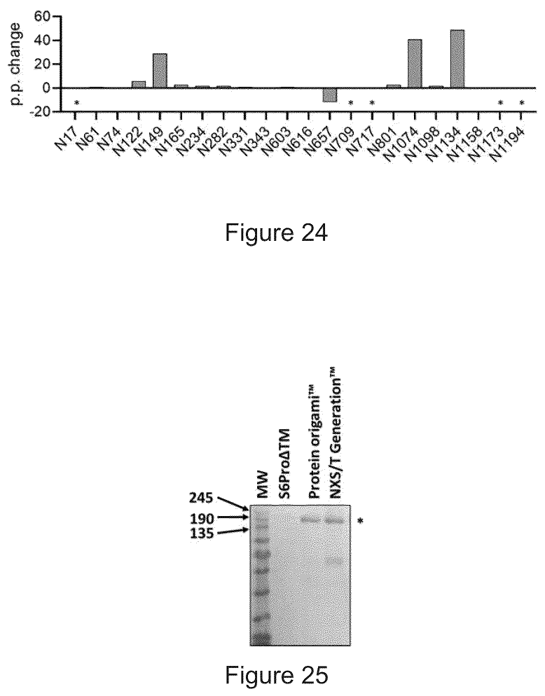

The increased aggregation witnessed for the “WT” SΔTM is consistent with observations for plant-produced HIV Envelope gp140 and MARV GPΔTM, as exemplified in Example 1, where aberrant glycosylation was associated with protein aggregation and inefficient folding and oligomerisation. Accordingly, the site-specific glycosylation of the “glyco-optimized” version of the SΔTM protein was determined after purification using GNL-affinity chromatography (FIG. 23), and the resulting data was compared to the “WT” antigen (FIG. 24).

Implementation of the integrated host and glyco-engineering approach to produce the “glyco-optimized” SΔTM yielded increased glycan occupancy at multiple sites across the protein (FIG. 24) which was associated with a concomitant improvement in protein folding and homogeneity (FIG. 22). This manifested as reduced aggregation and a more homogenous sample following gel filtration. Collectively, this data unequivocally demonstrates the utility of the integrated host and glyco-engineering (NXS/T Generation™) platform to produce complex glycoproteins in plants which would otherwise exceed the capacity of the endogenous machinery to support critical folding and glycosylation processes.

### Example 5

### Integrated Host and Glyco-Engineering Supports Production of a Well-Folded Prefusion Spike Trimer in Plants

Following the successful implementation of the NXS/T Generation™ platform to improve production of HIV Envelope gp140 and SARS-CoV-2 SΔTM, this was then applied to produce a stabilized prefusion SARS-CoV-2 spike trimer mimetic (S6ProΔTM) (SEQ ID NO:39). The antigen incorporates 6 proline mutations to stabilize the perfusion conformation of the molecule and to enhance expression. Additionally, the protein is prematurely truncated to remove the transmembrane and cytoplasmic regions rendering the resulting antigen soluble. The furin cleavage recognition sequence was replaced with a linker (GSAS) and polyhistidine and Strep-Tag Il affinity tags were incorporated at the C-terminus preceded by an HRC 3C site and GCN4 trimerization motif.

First it was demonstrated that co-expression of human calreticulin (Protein Origami™) was necessary to produce the antigen in plants, as had been shown for the analogous SΔTM in International Patent Publication No. WO 2021/220246 (FIG. 25). It was also further demonstrated that expression of the chaperone could be integrated with glyco-engineering to produce the spike protein (FIG. 25). Accordingly, the spike glycoprotein was transiently co-expressed in N. benthamiana alone, with human CRT (Protein Origami™), or with CRT and the glyco-engineering approaches that collectively constituted the NXS/T Generation™ platform (i.e integrated host and glyco-engineering). Crude plant homogenate was resolved by SDS-PAGE and western blotting was performed to detect expression of the recombinant S6ProΔTM antigen. In the absence of co-expressed chaperone, no expression of the glycoprotein was detected (S6ProΔTM; FIG. 25). Following the co-expression of human CRT (Protein Origami™; FIG. 25) the spike was easily detected as a product of ˜180 kDa. This indicates a substantially improvement in the production of the protein following chaperone co-expression, as exemplified in international patent application PA167643/US for other similarly complex glycoproteins. Specifically, it suggests that ectopic expression of the chaperone is necessary to produce the native spike at high levels in plants. Chaperone co-expression was also successfully integrated with the glyco-engineering approaches encompassed within the NXS/T Generation platform, as evidenced by the production of the expected ˜180 kDa product following western blotting (NXS/T Generation™; FIG. 25). This confirms that the impact of chaperone co-expression is not undermined by the simultaneous implementation of glyco-engineering, and that host and glyco-engineering approaches are complimentary.

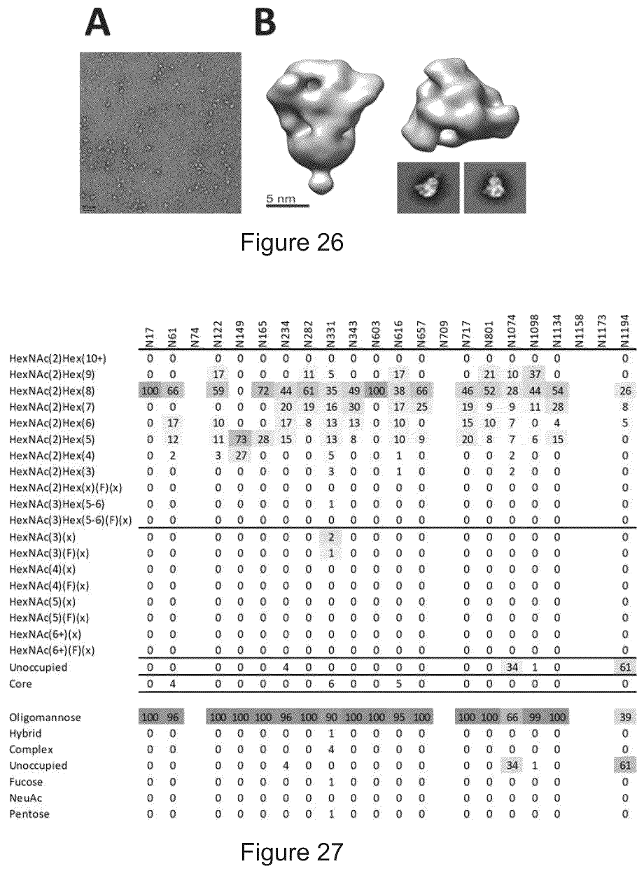

The antigen was purified by GNL-affinity chromatography and gel filtration, and pooled size exclusion chromatography fractions were subjected to negative stain transmission electron microscopy (FIG. 26A). This yielded a homogenous population of spike trimers with characteristic prefusion spike trimer morphology. Two-dimensional class averages derived from FIG. 26A further reinforced that the protein was well-folded and that the structure was consistent with the prefusion spike trimer (FIG. 26B). This data and the data in Example 4 collectively demonstrates that both host engineering (chaperone expression, Protein Origami™) and glyco-engineering are required to produce properly folded spike antigen in the system. This mirrors the observations for HIV Envelope gp140, exemplified in Example 3, which similarly requires remodeling of both the chaperone and glycosylation machinery to support the production of the protein in plants. Collectively, these examples confirm that integration of chaperone co-expression and glyco-engineering is necessary to produce well-folded and appropriately glycosylated complex glycoproteins in the system, and that these approaches support native-like oligomer formation.

The site-specific glycosylation of the purified trimer, produced by integrated host and glyco-engineering, was determined as before (FIG. 27). The antigen displayed high levels of glycan occupancy and negligible plant-specific glycan modifications, including plant-specific complex glycans and truncated (core) structures. Very low levels of core glycans were observed at N61 (4%), N331(6%) and N616 (5%) but these were drastically reduced compared to those observed in example 4. The glycans decorating the protein were almost exclusively high-mannose glycans.

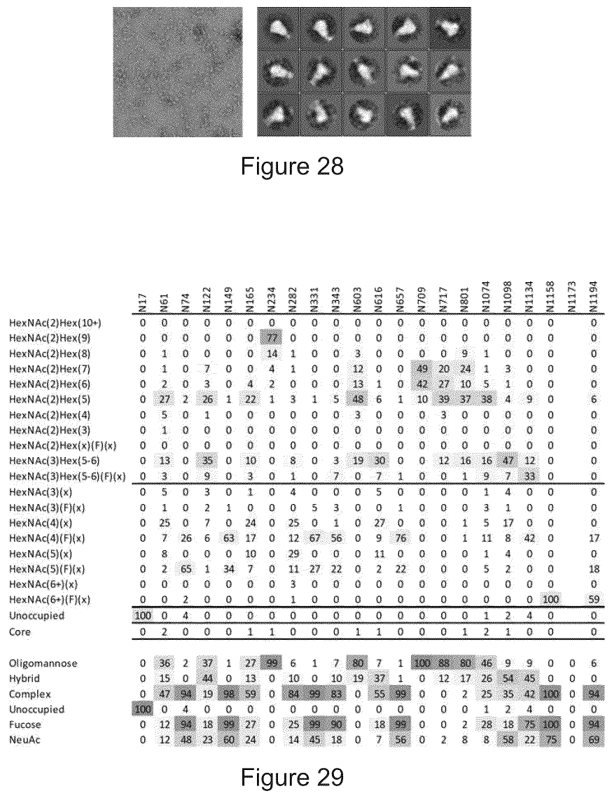

The matched antigen was also produced by transient transfection of HEK 293-F suspension cells to provide comparator material. The coding sequence of the gene was cloned into the pTHpCapR expression plasmid, exemplified in U.S. Pat. No. 8,460,933, and cells were transfected with 1 μg/ml plasmid DNA, at a density of 1×10⁶ cells/ml, using a 3:1 ratio of polyethylenimine:DNA. The culture media clarified by centrifugation at 2500 G, for 30 minutes, and then filtered a 0.45 M Stericup-GP device (Merck Millipore). Trimeric spike protein was purified with GNL-affinity chromatography and gel filtration, as described for the plant-produced S6ProΔTM. Negative stain electron microscopy revealed typical prefusion trimers which were well-folded and structurally comparable to the plant-derived material (FIG. 28).

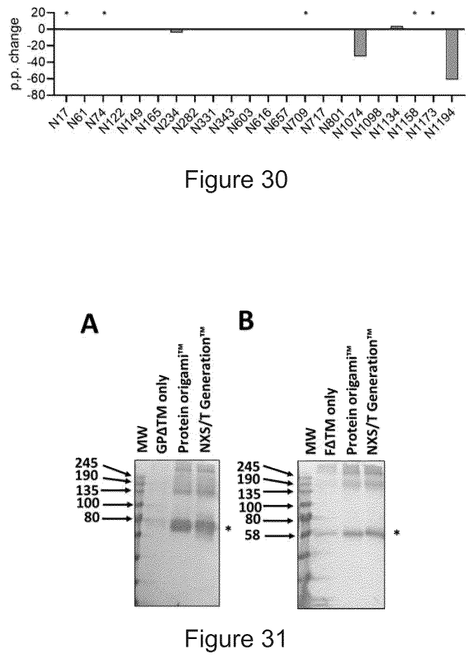

The site-specific glycosylation of the mammalian cell-produced SARS-CoV-2 S6ProΔTM was determined (FIG. 29). The antigen contained typical mammalian complex glycans decorated with core fucose, sialic acid and galactose extensions. A comparison of the site-specific glycan occupancy of the “glyco-optimized” and mammalian cell-produced S6ProΔTM antigens confirmed very similar levels of glycan occupancy (FIG. 30), contrasting to Example 4 where plant-produced spike protein contained notably lower levels of glycans across multiple sequins when produced in the absence of integrated host and glyco-engineering.

### Example 6

### Production of Viral Glycoproteins from Emerging Viruses Using Integrated Host and Glyco-Engineering (NXS/T Generation™)

Following the encouraging improvements that were observed with the NXS/T Generation production platform, we implemented this approach to produce viral glycoproteins from Ebola virus (UniProt Q05320), Nipah virus (Genbank AAK50544.1) and Lujo virus (UniProt C5ILC1) as examples of emerging viruses. All 3 glycoproteins were produced as soluble derivatives of the virion-associated protein by artificially truncating them to remove their respective transmembrane and cytoplasmic domains. This yielded antigens designated as EBOV GPΔTM (SEQ ID NO:41), NiV FΔTM (SEQ ID NO:43) and LUJV GP-CΔTM (SEQ ID NO:45) which corresponded to the soluble versions of the Ebola glycoprotein, the Nipah virus fusion glycoprotein and the Lujo virus GP-C glycoprotein, respectively. Additional stabilizing mutations were incorporated into the NiV FΔTM coding sequence (SEQ ID NO:43): 1114C, L104C, L172F and S191P. A heterologous GCN4 trimerization motif was also added at the C-terminus, followed by a linker peptide (GSGGSGGSG) and a polyhistidine tag (HHHHHHHH). Similarly, the EBOV GPΔTM (SEQ ID NO:41) contained T577P and K588F mutations to enhance trimer formation, and the native signal peptide was replaced with the signal peptide from tissue plasminogen activator protein. The protein also contained a C-terminal polyhistidine tag (HHHHHHHH), preceded by a flexible linker (GSGGSGGSG). The same linker and polyhistidine tag was added to the C-terminus of LUJV GP-CΔTM (SEQ ID NO:45). Lastly the Kozak sequence CCACC was added prior to the start of each sequence.

In each case, the soluble ectodomain of each respective glycoprotein was co-expressed with CRT in Nicotiana benthamiana wild type (Protein Origami™) or produced using integrated host and glyco-engineering in N. benthamiana ΔXF (NXS/T Generation™). Crude leaf lysate was resolved by SDS-PAGE and the proteins of interest were detected by western blotting. In the case of Ebola, the glycoprotein was barely detectable in the absence of the co-expressed chaperone (FIG. 31A; GPΔTM only). In contrast when calreticulin was co-expressed, the level of the antigen was substantially improved and the protein yielded a thick band at ˜80 kDa (FIG. 31A; Protein Origami™). This was also successfully combined with glyco-engineering, as evidenced by the successful detection of the desired product when these approaches were implemented (FIG. 31A; NXS/T Generation™). Similar observations arose with the Nipah fusion glycoprotein (SΔTM). Co-expression of CRT (protein Origami™) resulted in higher levels of production of the ˜58 kDa protein than when it was expressed alone (FΔTM only) (FIG. 31B). Once again the antigen was also successfully produced using the combination of approaches for integrated host and glyco-engineering (FIG. 31B; NXS/T Generation™). The latter appeared to result in an increase in size suggesting increased glycan occupancy.

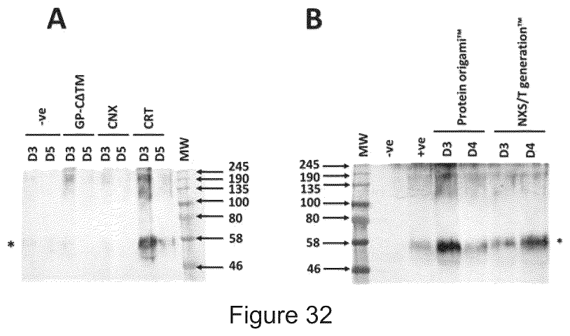

A similar approach was investigated for the LUJV GP-CΔTM antigen to demonstrate the utility of host engineering. Firstly, the antigen was co-expressed with human CRT and CNX (Protein Origami™) to demonstrate the impact of chaperone co-expression on their accumulation. Crude leaf homogenate, from 3 days (D3) and 5 days (D5) post agroinfiltration, were resolved by SDS-PAGE and subjected to western blotting (FIG. 32A). The expected ˜58 kDa protein was only detected following the co-expression of CRT. This was evident on both D3 and D5, although the intensity of the product was greatest at the earliest time point. Co-expression of GP-CΔTM and CRT was then combined with the integrated host and glyco-engineering approach that constitutes NXS/T Generation™. Once again, crude leaf lysate was resolved by SDS-PAGE and the protein of interest was detected by western blotting (FIG. 32B). The image demonstrates a size shift in the NXS/T Generation™ samples consistent with an increase in glycosylation. Due to the small size of the protein in this example the changes in glycosylation were apparent following western blotting.

1. A method of producing a heterologous polypeptide of interest in a plant cell, the method comprising:
(i) providing a first nucleic acid encoding a mammalian chaperone protein;
(ii) providing a second nucleic acid encoding a polypeptide which increases glycan occupancy;
(iii) providing a third nucleic acid which interferes with an enzyme which is responsible for the formation of truncated glycans in the plant cell;
(iv) providing a fourth nucleic acid encoding a heterologous polypeptide of interest;
(v) cloning the first, second, third and fourth nucleic acids into at least one expression vector adapted to express a polypeptide in a plant cell;
(vi) transforming or infiltrating a plant cell with the at least one expression vector of step (v);
(vii) co-expressing the polypeptide encoding the mammalian chaperone protein, the polypeptide which increases glycan occupancy, the nucleic acid which interferes with the enzyme responsible for the formation of truncated glycans and the heterologous polypeptide of interest in the plant cell; and
(viii) recovering the heterologous polypeptide of interest from the plant cell.

2. The method of claim 1, wherein the method results in at least one or more of the following:
(i) increased expression of the heterologous polypeptide of interest;
(ii) increased glycosylation efficiency of the heterologous polypeptide of interest;
(iii) a reduction in plant specific modifications of the heterologous polypeptide of interest;
(iv) a reduction in aggregation of the heterologous polypeptide of interest;
(v) increased folding efficiency of the heterologous polypeptide of interest; and/or
(vi) improved oligomerisation of the heterologous polypeptide of interest.

3. The method of claim 1, wherein the mammalian chaperone protein is at least one human chaperone protein selected from the group consisting of calnexin, calreticulin, GRP78/BiP, GRP94, GRP170, HSP47, ERp29, protein disulfide isomerase, peptidyl prolyl cis-trans-isomerase and ERp57.

4. The method of claim 3, wherein the human chaperone protein is selected from calnexin and/or calreticulin.

5. The method of claim 1, wherein the polypeptide which increases glycan occupancy is an oligosaccharyltransferase enzyme.

6. The method of claim 5, wherein the oligosaccharyltransferase enzyme is LmSTT3D from Leishmania major.

7. The method of claim 1, wherein the third nucleic acid is an RNAi expression cassette encoding an RNAi agent which interferes with a hexosaminidase 3 gene.

8. The method of claim 7, wherein the RNAi agent reduces the expression of the hexosaminidase 3 protein in the cell, thereby reducing the amount of truncated glycans produced in the cell.

9. The method of claim 1, wherein the plant cell is a Nicotiana benthamiana cell.

10. The method of claim 9, wherein the N. benthamiana cell is a glycosylation mutant lacking plant-specific N-glycan residues.

11. The method of claim 1, wherein the heterologous polypeptide of interest is a glycoprotein.

12. The method of claim 11, wherein the glycoprotein is a viral glycoprotein.

13. The method of claim 1, wherein the at least one expression vector includes promoters and/or other regulators, operably linked to the first, second, third and fourth nucleic acids.

14. A plant cell which is transformed with at least one expression vector, comprising:
a first nucleic acid encoding a mammalian chaperone protein;
a second nucleic acid encoding a polypeptide which increases glycan occupancy;
a third nucleic acid which interferes with an enzyme which is responsible for the formation of truncated glycans in the plant cell; and
a fourth nucleic acid encoding a heterologous polypeptide of interest.

15. The plant cell of claim 14, wherein the mammalian chaperone protein is at least one human chaperone protein selected from the group consisting of calnexin, calreticulin, GRP78/BiP, GRP94, GRP170, HSP47, ERp29, protein disulfide isomerase, peptidyl prolyl cis-trans-isomerase and ERp57.

16. The plant cell of claim 15, wherein the human chaperone protein is selected from calnexin and/or calreticulin.

17. The plant cell of claim 14, wherein the polypeptide which increases glycan occupancy is an oligosaccharyltransferase enzyme.

18. The plant cell of claim 17, wherein the oligosaccharyltransferase enzyme is LmSTT3D from Leishmania major.

19. The plant cell of claim 14, wherein the third nucleic acid is an RNAi expression cassette encoding an RNAi agent which interferes with a hexosaminidase 3 gene.

20. The plant cell of claim 19, wherein the RNAi agent reduces the expression of the hexosaminidase 3 protein in the cell, thereby reducing the amount of truncated glycans produced in the cell.

21. The plant cell of claim 14, wherein the heterologous polypeptide of interest is a glycoprotein.

22. The plant cell of claim 21, wherein the glycoprotein is a viral glycoprotein.

23. The plant cell of claim 14, wherein the at least one expression vector includes promoters and/or other regulators, operably linked to the first, second, third and fourth nucleic acids.

24. The plant cell of claim 14, wherein the plant cell is from a monocotyledonous or dicotyledonous plant.

25. The plant cell of claim 24, wherein the plant cell is from a plant selected from the group consisting of maize, rice, sorghum, wheat, cassava, barley, oats, rye, sweet potato, soybean, alfalfa, tobacco, sunflower, cotton, and canola.

26. The plant cell of claim 25, wherein the plant cell is from a tobacco plant.

27. The plant cell of claim 26, wherein the tobacco plant is Nicotiana benthamiana.

28. The plant cell of claim 27, wherein the N. benthamiana is a glycosylation mutant lacking plant-specific N-glycan residues.

29. A plant comprising the plant cell of claim 14.


SEQUENCE LISTING (45 sequences)

SEQ ID NO: 1 - DNA, 1266 bp, Artificial Sequence
  Modified Human Calreticulin
accggtatgctgctgagcgtgcctctgctgctggggctgctggggctggccgtcgccgaacctgctgtctacttcaaggaacagtttctggatggcgacggatggaccagccggtggatcgagagcaagcacaagtccgatttcggcaagtttgtgctgagctccggcaagttctatggcgatgaggagaaggacaagggcctgcagacaagccaggacgcccggttctacgccctgtctgccagcttcgagccattttccaacaagggccagaccctggtggtgcagttcacagtgaagcacgagcagaacatcgattgcggcggcggctatgtgaagctgtttcccaattccctggatcagaccgacatgcacggcgactctgagtacaacatcatgttcggcccagatatctgcggccccggcacaaagaaggtgcacgtgatctttaattataagggcaagaacgtgctgatcaataaggacatcaggtgtaaggacgatgagttcacccacctgtacacactgatcgtgcgccctgacaacacctatgaggtgaagatcgataattcccaggtggagtccggctctctggaggacgattgggattttctgccccctaagaagatcaaggaccctgatgcctctaagccagaggactgggatgagagggccaagatcgacgatcccacagacagcaagcctgaggactgggataagcccgagcacatccctgacccagatgccaagaagcccgaagactgggatgaggagatggatggcgagtgggagccacccgtgatccagaaccccgagtacaagggcgagtggaagcctagacagatcgataatccagactataagggcacctggatccacccagagatcgataaccccgagtactctcccgaccctagcatctacgcctatgataatttcggcgtgctgggcctggacctgtggcaggtgaagtctggcaccatcttcgacaactttctgatcacaaatgatgaggcctacgccgaggagtttggcaatgagacatggggcgtgacaaaggccgccgagaagcagatgaaggataagcaggacgaggagcagcggctgaaggaagaggaggaggacaagaagagaaaggaggaggaggaggccgaggataaggaggacgatgaggacaaggatgaggacgaggaggacgaggaggataaggaggaagatgaagaggaggatgtcccagggcaggcaaaagatgaactgtgactcgag

SEQ ID NO: 2 - protein, 417 aa, artificial sequence
  Modified Human Calreticulin
MLLSVPLLLGLLGLAVAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAKDEL

SEQ ID NO: 3 - DNA, 1791 bp, Artificial Sequence
  Modified Human Calnexin
accggtatggaggggaaatggctgctgtgcatgctgctggtgctgggaactgctattgtggaggctcacgacggacacgatgacgatgtgattgacatcgaggacgatctggacgatgtgatcgaggaggtggaggacagcaagcctgataccacagccccccctagctccccaaaggtgacctacaaggcccccgtgcctacaggcgaggtgtatttcgccgactcctttgataggggcaccctgagcggatggatcctgtccaaggccaagaaggacgatacagacgatgagatcgccaagtacgacggcaagtgggaagtggaggagatgaaggagtctaagctgcctggcgataagggcctggtgctgatgtcccgggccaagcaccacgccatctctgccaagctgaataagccattcctgtttgacaccaagcccctgatcgtgcagtacgaggtgaacttccagaatggcatcgagtgcggcggcgcctatgtgaagctgctgtccaagacaccagagctgaatctggaccagttccacgataagaccccttacacaatcatgtttggcccagacaagtgtggcgaggattataagctgcacttcatctttagacacaagaacccaaagaccggcatctatgaggagaagcacgccaagaggcccgacgccgatctgaagacctacttcacagacaagaagacccacctgtatacactgatcctgaacccagacaattcttttgagatcctggtggatcagtccgtggtgaactctggcaatctgctgaacgatatgaccccacccgtgaatcccagcagggagatcgaggaccccgaggatcgcaagcctgaggactgggatgagcggcccaagatcccagacccagaggcagtgaagcctgacgattgggacgaggatgcccctgccaagatcccagatgaggaggccacaaagcccgagggctggctggacgatgagcctgagtacgtgcctgacccagatgccgagaagcccgaggactgggatgaggacatggatggcgagtgggaggccccacagatcgcaaacccaagatgcgagagcgcccctggatgtggcgtgtggcagaggcctgtgatcgacaacccaaattacaagggcaagtggaagcctccaatgatcgataatccatcctatcagggcatctggaagccccgcaagatccccaaccctgacttctttgaggatctggagcccttccggatgacccctttttctgccatcggcctggagctgtggtctatgacaagcgacatcttctttgataacttcatcatctgcgccgaccggagaatcgtggacgattgggccaacgacggatggggcctgaagaaggcagcagatggagcagcagagccaggagtggtgggacagatgatcgaggcagcagaggagcggccctggctgtgggtggtgtacatcctgaccgtggccctgcccgtgttcctggtcatcctgttctgctgttctggcaagaagcagaccagcggcatggagtataagaagacagacgccccacagcccgatgtgaaagaggaggaggaggagaaggaggaggagaaggacaagggcgatgaggaggaggagggcgaggagaagctggaggagaagcagaagagcgacgccgaggaggatggcggcacagtgtcccaggaggaggaggaccggaagcctaaggcagaagaagacgaaatcctgaatcggtcaccaagaaatagaaaaccacggagggaatgactcgag

SEQ ID NO: 4 - protein, 592 aa, Artificial Sequence
  Modified Human Calnexin
MEGKWLLCMLLVLGTAIVEAHDGHDDDVIDIEDDLDDVIEEVEDSKPDTTAPPSSPKVTYKAPVPTGEVYFADSFDRGTLSGWILSKAKKDDTDDEIAKYDGKWEVEEMKESKLPGDKGLVLMSRAKHHAISAKLNKPFLFDTKPLIVQYEVNFQNGIECGGAYVKLLSKTPELNLDQFHDKTPYTIMFGPDKCGEDYKLHFIFRHKNPKTGIYEEKHAKRPDADLKTYFTDKKTHLYTLILNPDNSFEILVDQSVVNSGNLLNDMTPPVNPSREIEDPEDRKPEDWDERPKIPDPEAVKPDDWDEDAPAKIPDEEATKPEGWLDDEPEYVPDPDAEKPEDWDEDMDGEWEAPQIANPRCESAPGCGVWQRPVIDNPNYKGKWKPPMIDNPSYQGIWKPRKIPNPDFFEDLEPFRMTPFSAIGLELWSMTSDIFFDNFIICADRRIVDDWANDGWGLKKAADGAAEPGVVGQMIEAAEERPWLWVVYILTVALPVFLVILFCCSGKKQTSGMEYKKTDAPQPDVKEEEEEKEEEKDKGDEEEEGEEKLEEKQKSDAEEDGGTVSQEEEDRKPKAEEDEILNRSPRNRKPRRE

SEQ ID NO: 5 - DNA, 2583 bp, Artificial sequence
  Leishmania major LmSTT3D
tctagaatgggtaagagaaagggaaacagcctgggagattctggttctgctgctactgcttcaagagaggcttctgctcaagctgaggatgctgcttctcagactaagactgctagccctcctgctaaggtgatccttttgcctaagaccctgaccgatgagaaggattttatcggtatcttccctttcccattctggcctgtgcatttcgtgcttactgtggtggctcttttcgtgttggctgcttcttgcttccaggctttcaccgtgaggatgatctctgtgcagatctacggttacctgatccacgagttcgatccatggttcaactatagggctgctgagtacatgtctacccacggttggtctgctttcttcagctggttcgattacatgagctggtatcctcttggtaggcctgtgggttctactacctatcctggacttcagcttaccgctgtggctattcatagagctttggctgctgctggtatgcctatgtctctgaacaatgtgtgcgtgctgatgcctgcttggtttggtgctattgctaccgctactcttgctttctgtacctacgaggcttcaggttctactgttgctgctgcagctgctgctctgagcttctctattattcctgctcacctgatgagatccatggctggtgagttcgataacgagtgcattgctgtggctgctatgctgcttactttctactgctgggtgagaagccttaggaccagatcttcttggcctattggtgtgcttaccggtgttgcttacggttacatggctgcagcttggggaggttacattttcgtgctgaacatggtggctatgcacgctggaatcagctctatggttgattgggctaggaatacctacaacccatctctgcttagggcttacaccctgttctacgttgtgggaaccgctattgctgtttgtgtgcctcctgttggtatgagccctttcaagtctcttgagcagcttggtgctctgcttgtgcttgttttcttgtgcggacttcaggtgtgcgaggttttgagagctagagctggtgttgaggttaggtccagggctaacttcaagatcagggtgagggtgttctcagtgatggctggtgttgctgctctggctatttctgtgcttgctcctactggttacttcggtcctttgtctgttagggttagggctctgttcgttgagcataccaggactggtaaccctctggttgattctgttgctgagcatcagcctgcttctccagaggctatgtgggcttttcttcatgtgtgcggtgtgacttggggtctgggttctatcgttcttgctgtgtctaccttcgtgcactacagcccttctaaggtgttctggcttctgaactctggtgctgtgtactacttctctaccaggatggctaggcttcttctgttgtctggacctgctgcttgcctgtctactggtattttcgtgggaaccatccttgaggctgctgtgcagctttcattctgggattctgatgctaccaaggctaagaaacagcaaaagcaggctcagaggcatcagagaggtgctggtaagggttctggaagggatgatgctaagaatgctactaccgctagggctttctgtgatgtgttcgctggttcttctcttgcttggggtcataggatggtgctgtctattgcaatgtgggctcttgtgactaccaccgctgtgtctttcttctcctccgaatttgcttcccactccaccaagttcgctgagcagtcatctaaccctatgatcgtgttcgcagctgtggtgcagaatagggctactggaaagcctatgaacctgctggtggatgattacctgaaggcttacgagtggctgagggattctactccagaggatgctagagttctggcatggtgggattacggataccagattaccggtatcggtaacaggacctctctggctgatggtaatacttggaaccacgagcacattgctaccatcggtaagatgcttaccagccctgttgttgaggctcactctcttgttaggcacatggctgattacgtgctgatttgggctggtcagtctggtgatctgatgaagtctcctcacatggctaggatcggtaactccgtgtaccacgatatctgccctgatgatcctctttgtcagcagttcggtttccataggaacgattactctaggcctacccctatgatgagggcttctcttctttacaacctgcacgaggcaggtaaaagaaaaggtgtgaaggtgaaccctagcctgttccaagaggtgtacagctctaagtacggtctggtgaggatcttcaaggtgatgaacgtgagcgctgagagcaagaagtgggttgcagatcctgctaatagggtgtgccatcctcctggttcttggatttgtcctggtcagtaccctccagctaaagaaattcaagagatgctggctcacagggtgccattcgatcaggttaccaacgctgataggaagaacaacgttggaagctatcaagaggagtacatgagaagaatgagggaatccgagaacagaaggggatcc

SEQ ID NO: 6 - protein, 859 aa, Artificial sequence
  Leishmania major LmSTT3D
MGKRKGNSLGDSGSAATASREASAQAEDAASQTKTASPPAKVILLPKTLTDEKDFIGIFPFPFWPVHFVLTVVALFVLAASCFQAFTVRMISVQIYGYLIHEFDPWFNYRAAEYMSTHGWSAFFSWFDYMSWYPLGRPVGSTTYPGLQLTAVAIHRALAAAGMPMSLNNVCVLMPAWFGAIATATLAFCTYEASGSTVAAAAAALSFSIIPAHLMRSMAGEFDNECIAVAAMLLTFYCWVRSLRTRSSWPIGVLTGVAYGYMAAAWGGYIFVLNMVAMHAGISSMVDWARNTYNPSLLRAYTLFYVVGTAIAVCVPPVGMSPFKSLEQLGALLVLVFLCGLQVCEVLRARAGVEVRSRANFKIRVRVFSVMAGVAALAISVLAPTGYFGPLSVRVRALFVEHTRTGNPLVDSVAEHQPASPEAMWAFLHVCGVTWGLGSIVLAVSTFVHYSPSKVFWLLNSGAVYYFSTRMARLLLLSGPAACLSTGIFVGTILEAAVQLSFWDSDATKAKKQQKQAQRHQRGAGKGSGRDDAKNATTARAFCDVFAGSSLAWGHRMVLSIAMWALVTTTAVSFFSSEFASHSTKFAEQSSNPMIVFAAVVQNRATGKPMNLLVDDYLKAYEWLRDSTPEDARVLAWWDYGYQITGIGNRTSLADGNTWNHEHIATIGKMLTSPVVEAHSLVRHMADYVLIWAGQSGDLMKSPHMARIGNSVYHDICPDDPLCQQFGFHRNDYSRPTPMMRASLLYNLHEAGKRKGVKVNPSLFQEVYSSKYGLVRIFKVMNVSAESKKWVADPANRVCHPPGSWICPGQYPPAKEIQEMLAHRVPFDQVTNADRKNNVGSYQEEYMRRMRESENRRGS

SEQ ID NO: 7 - DNA, 220 bp, Artificial Sequence
  HEXO3RNAi sense strand
gcaaaaacagtttatggggcgttgcatggtttgcagacatttagtcaagtatgccattttaactttacaaccagaacaattgaagttcatcaagttccatggaccatagttgatcgaccaagattctcttatcgagggcttttaattgatacttcccgtcactatctgccgttgcctgtgatattgaaggttatcgattcaatggcttatgcaaaactga

SEQ ID NO: 8 - DNA, 220 bp, Artificial Sequence
  HEXO3RNAi antisense strand
tcagttttgcataagccattgaatcgataaccttcaatatcacaggcaacggcagatagtgacgggaagtatcaattaaaagccctcgataagagaatcttggtcgatcaactatggtccatggaacttgatgaacttcaattgttctggttgtaaagttaaaatggcatacttgactaaatgtctgcaaaccatgcaacgccccataaactgtttttgc

SEQ ID NO: 9 - DNA, 1984 bp, Artificial Sequence
  Modified HIV envelope gp140 for expression in mammalian cells
atggatgctatgaagcggggactctgttgcgtgctgctgctgtgcggagccgtgttcgtgtcaccctctgccggagggctgtgggtcactgtctactatggcgtgcctgtctggagagaggccaagaccacactgttctgcgcttccgatgcaaagtcttacgaaaaagaggtgcacaacgtctgggccacacatgcttgcgtgccaactgaccccaaccctcaggaactggtgctgaagaatgtcaccgagaactttaatatgtggaaaaatgacatggtggatcagatgcacgaggatatcattagtctgtgggaccagtcactgaagccctgcgtgaaactgacacctctgtgcgtcactctgaactgtagcgatgcaaaggtgaacattaatgccacatacaatggcactcgcgaggaaatcaaaaactgttccttcaatgcaactaccgaactgagggacaagaagaagaaggagtacgccctgttttatcgcctggacatcgtgcccctgaacaaggaagggaacaataacagtgagtatcggctgattaactgcaataccagcgtgattacccaggcctgtcctaaagtcaccttcgatccaattcccatccactactgcgcaccagccggatatgctattctgaagtgtaacaacaaaacttttaacgggaccggaccctgcaataacgtgtctacagtccagtgtactcatggcatcaagcctgtggtctcaacccagctgctgctgaatgggagcctggccgaggaagagatcattatcagaagcgagaacctgaccgacaatgtgaagacaattatcgtccacctgaacgaatccgtggagattaattgcaccaggccaaacaacaacacacgaaaatctattcggatcggaccaggacagaccttctacgcaacaggggacattatcggagatatcaggcaggctcattgtaacatttctgaaatcaagtgggagaaaaccctgcagcgcgtgagtgaaaagctgcgagagcacttcaacaaaacaatcatctttaatcagagctccggcggggacctggaaatcacaactcattcattcaactgcggaggcgagttcttttactgtaacactagcgatctgttctttaataagacctttgacgagacctattccacaggctcaaacagcactaattctaccattacactgccatgccgaatcaaacagattatcaacatgtggcaggaagtgggccgggcaatgtatgccagccccattgccggagagatcacctgtaagtccaatatcactggactgctgctgaccagagatgggggaggcaacaattctactgaagagacctttaggcccgggggaggcaacatgagagacaattggaggagcgaactgtacaagtataaagtggtcgaggtgaagcctctgggaatcgcaccaaccgaggcccggagaagggtggtccagcagggcggtggaggctcaggtggaggcggatccgctgtggtcggactgggagcagtgttcctggggtttctgggaactgctggcagcaccatgggagccgcttccattactctgaccgtgcaggcacgccagctgctgtctggcatcgtccagcagcagagtaacctgctgcgggctcctgaagcacagcagcatatgctgcagctgaccgtgtgggggattaagcagctgcaggcccgggtcctggctatcgagagatacctgaaggatcagcagctgctggggatgtggggatgcagtggcaaactgatttgcaccacaaacgtgtactggaacagcagctggtccaacaagacatataatgaaatctgggacaacatgacttggatgcagtgggaccgcgagatcgataactacacagacactatctataaactgctggaagtctcacagaaacagcaggagtcaaatgaaaaggacctgctggcactggatgcggccgcatgat

SEQ ID NO: 10 - protein, 658 aa, Artificial Sequence
  Modified HIV envelope gp140 expressed in mammalian cells
MDAMKRGLCCVLLLCGAVFVSPAGLWVTVYYGVPVWREAKTTLFCASDAKSYEKEVHNVWATHACVPTDPNPQELVLKNVTENFNMWKNDMVDQMHEDIISLWDQSLKPCVKLTPLCVTLNCSDAKVNINATYNGTREEIKNCSFNATTELRDKKKKEYALFYRLDIVPLNKEGNNNSEYRLINCNTSVITQACPKVTFDPIPIHYCAPAGYAILKCNNKTFNGTGPCNNVSTVQCTHGIKPVVSTQLLLNGSLAEEEIIIRSENLTDNVKTIIVHLNESVEINCTRPNNNTRKSIRIGPGQTFYATGDIIGDIRQAHCNISEIKWEKTLQRVSEKLREHFNKTIIFNQSSGGDLEITTHSFNCGGEFFYCNTSDLFFNKTFDETYSTGSNSTNSTITLPCRIKQIINMWQEVGRAMYASPIAGEITCKSNITGLLLTRDGGGNNSTEETFRPGGGNMRDNWRSELYKYKVVEVKPLGIAPTEARRRVVQQGGGGSGGGGSAVVGLGAVFLGFLGTAGSTMGAASITLTVQARQLLSGIVQQQSNLLRAPEAQQHMLQLTVWGIKQLQARVLAIERYLKDQQLLGMWGCSGKLICTTNVYWNSSWSNKTYNEIWDNMTWMQWDREIDNYTDTIYKLLEVSQKQQESNEKDLLALDAAA

SEQ ID NO: 11 - DNA, 2018 bp, Artificial Sequence
  Modified HIV envelope gp140 for expression in plant cells
tcatgaaagcttaccggtgccaccatggagtggtcttggatcttcctgtttctgctgagcgggactgctggagtgcattcttccggagggctgtgggtcactgtctactatggcgtgcctgtctggagagaggccaagaccacactgttctgcgcttccgatgcaaagtcttacgaaaaagaggtgcacaacgtctgggccacacatgcttgcgtgccaactgaccccaaccctcaggaactggtgctgaagaatgtcaccgagaactttaatatgtggaaaaatgacatggtggatcagatgcacgaggatatcattagtctgtgggaccagtcactgaagccctgcgtgaaactgacacctctgtgcgtcactctgaactgtagcgatgcaaaggtgaacattaatgccacatacaatggcactcgcgaggaaatcaaaaactgttccttcaatgcaactaccgaactgagggacaagaagaagaaggagtacgccctgttttatcgcctggacatcgtgcccctgaacaaggaagggaacaataacagtgagtatcggctgattaactgcaataccagcgtgattacccaggcctgtcctaaagtcaccttcgatccaattcccatccactactgcgcaccagccggatatgctattctgaagtgtaacaacaaaacttttaacgggaccggaccctgcaataacgtgtctacagtccagtgtactcatggcatcaagcctgtggtctcaacccagctgctgctgaatgggagcctggccgaggaagagatcattatcagaagcgagaacctgaccgacaatgtgaagacaattatcgtccacctgaacgaatccgtggagattaattgcaccaggccaaacaacaacacacgaaaatctattcggatcggaccaggacagaccttctacgcaacaggggacattatcggagatatcaggcaggctcattgtaacatttctgaaatcaagtgggagaaaaccctgcagcgcgtgagtgaaaagctgcgagagcacttcaacaaaacaatcatctttaatcagagctccggcggggacctggaaatcacaactcattcattcaactgcggaggcgagttcttttactgtaacactagcgatctgttctttaataagacctttgacgagacctattccacaggctcaaacagcactaattctaccattacactgccatgccgaatcaaacagattatcaacatgtggcaggaagtgggccgggcaatgtatgccagccccattgccggagagatcacctgtaagtccaatatcactggactgctgctgaccagagatgggggaggcaacaattctactgaagagacctttaggcccgggggaggcaacatgagagacaattggaggagcgaactgtacaagtataaagtggtcgaggtgaagcctctgggaatcgcaccaaccgaggcccggagaagggtggtccagcagggcggtggaggctcaggtggaggcggatccgctgtggtcggactgggagcagtgttcctggggtttctgggaactgctggcagcaccatgggagccgcttccattactctgaccgtgcaggcacgccagctgctgtctggcatcgtccagcagcagagtaacctgctgcgggctcctgaagcacagcagcatatgctgcagctgaccgtgtgggggattaagcagctgcaggcccgggtcctggctatcgagagatacctgaaggatcagcagctgctggggatgtggggatgcagtggcaaactgatttgcaccacaaacgtgtactggaacagcagctggtccaacaagacatataatgaaatctgggacaacatgacttggatgcagtgggaccgcgagatcgataactacacagacactatctataaactgctggaagtctcacagaaacagcaggagtcaaatgaaaaggacctgctggcactggatgcggccgcatgatttttctgaattctagactcgag

SEQ ID NO: 12 - protein, 656 aa, Artificial Sequence
  Modified HIV envelope gp140 expressed in plant cells
MEWSWIFLFLLSGTAGVHSSGGLWVTVYYGVPVWREAKTTLFCASDAKSYEKEVHNVWATHACVPTDPNPQELVLKNVTENFNMWKNDMVDQMHEDIISLWDQSLKPCVKLTPLCVTLNCSDAKVNINATYNGTREEIKNCSFNATTELRDKKKKEYALFYRLDIVPLNKEGNNNSEYRLINCNTSVITQACPKVTFDPIPIHYCAPAGYAILKCNNKTFNGTGPCNNVSTVQCTHGIKPVVSTQLLLNGSLAEEEIIIRSENLTDNVKTIIVHLNESVEINCTRPNNNTRKSIRIGPGQTFYATGDIIGDIRQAHCNISEIKWEKTLQRVSEKLREHFNKTIIFNQSSGGDLEITTHSFNCGGEFFYCNTSDLFFNKTFDETYSTGSNSTNSTITLPCRIKQIINMWQEVGRAMYASPIAGEITCKSNITGLLLTRDGGGNNSTEETFRPGGGNMRDNWRSELYKYKVVEVKPLGIAPTEARRRVVQQGGGGSGGGGSAVVGLGAVFLGFLGTAGSTMGAASITLTVQARQLLSGIVQQQSNLLRAPEAQQHMLQLTVWGIKQLQARVLAIERYLKDQQLLGMWGCSGKLICTTNVYWNSSWSNKTYNEIWDNMTWMQWDREIDNYTDTIYKLLEVSQKQQESNEKDLLALDAAA

SEQ ID NO: 13 - DNA, 2007 bp, Artificial Sequence
  Marburg viral glycoprotein for expression in mammalian cells
aagcttccaccatggatgcaatgaagagagggctctgctgtgtgctgctgctgtgtggagcagtcttcgtttcgcccagcgctctgcctattctggaaatcgcctcaaacaatcagcctcagaacgtggacagcgtgtgctctggcaccctgcagaagacagaggatgtgcacctgatgggcttcaccctgagcggccagaaggtggcagactccccactggaggcctctaagaggtgggcctttcgcaccggcgtgccacctaagaatgtggagtacaccgagggcgaggaggccaagacatgctataacatctccgtgaccgatcccagcggcaagtccctgctgctggacccacccaccaatatccgggattacccaaagtgtaagacaatccaccacatccagggacagaacccacacgcacagggaatcgccctgcacctgtggggcgccttctttctgtacgaccggatcgccagcaccacaatgtatagaggcaaggtgttcaccgagggcaatatcgccgccatgatcgtgaacaagacagtgcacaagatgatctttagccggcagggccagggctacagacacatgaatctgacatccaccaacaagtattggaccagctccaatggcacacagaccaacgatacaggctgcttcggcgccctgcaggagtataattctaccaagaaccagacatgtgcaccaagcaagatccctccaccactgccaaccgcccggcctgagatcaagctgacaagcacccctacagacgccacaaagctgaacaccacagacccatctagcgacgatgaggatctggccacctccggctctggctccggagagagagagcctcacaccacatccgatgccgtgaccaagcagggcctgtcctctaccatgcctccaacacctagcccacagccatccacacctcagcagggaggcaacaataccaatcactcccaggacgccgtgacagagctggataagaacaataccacagcccagccatctatgccccctcacaacaccacaaccatcagcaccaacaatacatccaagcacaatttttctaccctgagcgcccccctgcagaacacaaccaacgacaatacccagtctaccatcacagagaatgagcagacatccgccccctctatcacaaccctgccacccaccggcaaccctacaaccgccaagagcacaagctccaagaagggccctgccacaaccgccccaaatacaaccaacgagcacttcaccagccctccacccaccccatctagcacagcccagcacctggtgtactttggcggtggaggctcaggtggaggcggatcctctatcctgtggagggagggcgacatgttcccctttctggatggcctgatcaatgcccctatcgatttcgatcctgtgccaaacaccaagacaatctttgacgagagctcctctagcggcgcatccgccgaggaggatcagcacgcctctccaaatatcagcctgaccctgtcctacttccccaacatcaatgagaacacagcctattctggcgagaatgagaacgactgcgatgccgagctgaggatctggtccgtgcaggaggacgatctggcagcaggcctgtcttggattcccttcttcggaccaggaatcgagggcctgtataccgccgtgctgatcaagaatcagaacaacctggtgtgcaggctgcggagactggcaaaccagaccgccaagtctctggagctgctgctgagggtgacaaccgaggagcgcaccttcagcctgatcaataggcacgccatcgactttctgctgaccagatggggaggaacatgcaaggtgctgggccctgactgctgtatcggcatcgaggatctgtctaagaacatcagcgagcagatcgaccagatcaagaaggatgagcagaaggagggaaccggctggggcctgggcggcaagtggtggacatccgattgatttttctgaattc

SEQ ID NO: 14 - protein, 660 aa, Artificial Sequence
  Marburg viral glycoprotein expressed in mammalian cells
MDAMKRGLCCVLLLCGAVFVSPSALPILEIASNNQPQNVDSVCSGTLQKTEDVHLMGFTLSGQKVADSPLEASKRWAFRTGVPPKNVEYTEGEEAKTCYNISVTDPSGKSLLLDPPTNIRDYPKCKTIHHIQGQNPHAQGIALHLWGAFFLYDRIASTTMYRGKVFTEGNIAAMIVNKTVHKMIFSRQGQGYRHMNLTSTNKYWTSSNGTQTNDTGCFGALQEYNSTKNQTCAPSKIPPPLPTARPEIKLTSTPTDATKLNTTDPSSDDEDLATSGSGSGEREPHTTSDAVTKQGLSSTMPPTPSPQPSTPQQGGNNTNHSQDAVTELDKNNTTAQPSMPPHNTTTISTNNTSKHNFSTLSAPLQNTTNDNTQSTITENEQTSAPSITTLPPTGNPTTAKSTSSKKGPATTAPNTTNEHFTSPPPTPSSTAQHLVYFGGGGSGGGGSSILWREGDMFPFLDGLINAPIDFDPVPNTKTIFDESSSSGASAEEDQHASPNISLTLSYFPNINENTAYSGENENDCDAELRIWSVQEDDLAAGLSWIPFFGPGIEGLYTAVLIKNQNNLVCRLRRLANQTAKSLELLLRVTTEERTFSLINRHAIDFLLTRWGGTCKVLGPDCCIGIEDLSKNISEQIDQIKKDEQKEGTGWGLGGKWWTSD

SEQ ID NO: 15 - DNA, 2006 bp, Artificial Sequence
  Marburg viral glycoprotein for expression in plant cells
accggtaagcttccaccatggagtggagttggatctttctgttcctgctgtcagggaccgctggagtgcatagcgctctgcctattctggaaatcgcctcaaacaatcagcctcagaacgtggacagcgtgtgctctggcaccctgcagaagacagaggatgtgcacctgatgggcttcaccctgagcggccagaaggtggcagactccccactggaggcctctaagaggtgggcctttcgcaccggcgtgccacctaagaatgtggagtacaccgagggcgaggaggccaagacatgctataacatctccgtgaccgatcccagcggcaagtccctgctgctggacccacccaccaatatccgggattacccaaagtgtaagacaatccaccacatccagggacagaacccacacgcacagggaatcgccctgcacctgtggggcgccttctttctgtacgaccggatcgccagcaccacaatgtatagaggcaaggtgttcaccgagggcaatatcgccgccatgatcgtgaacaagacagtgcacaagatgatctttagccggcagggccagggctacagacacatgaatctgacatccaccaacaagtattggaccagctccaatggcacacagaccaacgatacaggctgcttcggcgccctgcaggagtataattctaccaagaaccagacatgtgcaccaagcaagatccctccaccactgccaaccgcccggcctgagatcaagctgacaagcacccctacagacgccacaaagctgaacaccacagacccatctagcgacgatgaggatctggccacctccggctctggctccggagagagagagcctcacaccacatccgatgccgtgaccaagcagggcctgtcctctaccatgcctccaacacctagcccacagccatccacacctcagcagggaggcaacaataccaatcactcccaggacgccgtgacagagctggataagaacaataccacagcccagccatctatgccccctcacaacaccacaaccatcagcaccaacaatacatccaagcacaatttttctaccctgagcgcccccctgcagaacacaaccaacgacaatacccagtctaccatcacagagaatgagcagacatccgccccctctatcacaaccctgccacccaccggcaaccctacaaccgccaagagcacaagctccaagaagggccctgccacaaccgccccaaatacaaccaacgagcacttcaccagccctccacccaccccatctagcacagcccagcacctggtgtactttggcggtggaggctcaggtggaggcggatcctctatcctgtggagggagggcgacatgttcccctttctggatggcctgatcaatgcccctatcgatttcgatcctgtgccaaacaccaagacaatctttgacgagagctcctctagcggcgcatccgccgaggaggatcagcacgcctctccaaatatcagcctgaccctgtcctacttccccaacatcaatgagaacacagcctattctggcgagaatgagaacgactgcgatgccgagctgaggatctggtccgtgcaggaggacgatctggcagcaggcctgtcttggattcccttcttcggaccaggaatcgagggcctgtataccgccgtgctgatcaagaatcagaacaacctggtgtgcaggctgcggagactggcaaaccagaccgccaagtctctggagctgctgctgagggtgacaaccgaggagcgcaccttcagcctgatcaataggcacgccatcgactttctgctgaccagatggggaggaacatgcaaggtgctgggccctgactgctgtatcggcatcgaggatctgtctaagaacatcagcgagcagatcgaccagatcaagaaggatgagcagaaggagggaaccggctggggcctgggcggcaagtggtggacatccgattgatttttctgaattctcgag

SEQ ID NO: 16 - protein, 656 aa, Artificial sequence
  Marburg viral glycoprotein expressed in plant cells
MEWSWIFLFLLSGTAGVHSALPILEIASNNQPQNVDSVCSGTLQKTEDVHLMGFTLSGQKVADSPLEASKRWAFRTGVPPKNVEYTEGEEAKTCYNISVTDPSGKSLLLDPPTNIRDYPKCKTIHHIQGQNPHAQGIALHLWGAFFLYDRIASTTMYRGKVFTEGNIAAMIVNKTVHKMIFSRQGQGYRHMNLTSTNKYWTSSNGTQTNDTGCFGALQEYNSTKNQTCAPSKIPPPLPTARPEIKLTSTPTDATKLNTTDPSSDDEDLATSGSGSGEREPHTTSDAVTKQGLSSTMPPTPSPQPSTPQQGGNNTNHSQDAVTELDKNNTTAQPSMPPHNTTTISTNNTSKHNFSTLSAPLQNTTNDNTQSTITENEQTSAPSITTLPPTGNPTTAKSTSSKKGPATTAPNTTNEHFTSPPPTPSSTAQHLVYFGGGGSGGGGSSILWREGDMFPFLDGLINAPIDFDPVPNTKTIFDESSSSGASAEEDQHASPNISLTLSYFPNINENTAYSGENENDCDAELRIWSVQEDDLAAGLSWIPFFGPGIEGLYTAVLIKNQNNLVCRLRRLANQTAKSLELLLRVTTEERTFSLINRHAIDFLLTRWGGTCKVLGPDCCIGIEDLSKNISEQIDQIKKDEQKEGTGWGLGGKWWTSD

SEQ ID NO: 17 - DNA, 68 bp, Artificial sequence
  TPA leader sequence for expression with modified HIV Env gp140
atggatgctatgaagcggggactctgttgcgtgctgctgctgtgcggagccgtgttcgtgtcaccctc

SEQ ID NO: 18 - DNA, 66 bp, Artificial sequence
  TPA leader sequence for expression with modified Marburg viral glycoprotein
atggatgcaatgaagagagggctctgctgtgtgctgctgctgtgtggagcagtcttcgtttcgccc

SEQ ID NO: 19 - DNA, 66 bp, Artificial sequence
  TPA leader sequence for expression with cleaved SOSIP.664
atggatgctatgaaaagggggctgtgctgcgtcctgctgctgtgcggggctgtcttcgtgtcacca

SEQ ID NO: 20 - protein, 22 aa, Artificial sequence
  Amino acid sequence for tissue plasminogen activator leader sequence
MDAMKRGLCCVLLLCGAVFVSP

SEQ ID NO: 21 - DNA, 57 bp, Artificial sequence
  Murine monoclonal leader peptide heavy chain for the modified HIV env gp140
atggagtggtcttggatcttcctgtttctgctgagcgggactgctggagtgcattct

SEQ ID NO: 22 - DNA, 57 bp, Artificial sequence
  Murine monoclonal leader peptide heavy chain for the Marburg viral glycoprotein
atggagtggagttggatctttctgttcctgctgtcagggaccgctggagtgcatagc

SEQ ID NO: 23 - DNA, 66 bp, Artificial sequence; identical to SEQ ID NO: 19
  Murine monoclonal leader peptide heavy chain for the modified Epstein-Barr virus gp350

SEQ ID NO: 24 - protein, 19 aa, Artificial sequence
  Amino acid sequence for the murine monoclonal leader peptide heavy chain
MEWSWIFLFLLSGTAGVHS

SEQ ID NO: 25 - protein, 4 aa, Artificial sequence
  Amino acid sequence of the furin cleavage site for the modified HIV env gp140 polypeptide
RERR

SEQ ID NO: 26 - protein, 4 aa, Artificial sequence
  Amino acid sequence of the furin cleavage site for the modified Marburg viral glycoprotein
RRKR

SEQ ID NO: 27 - DNA, 30 bp, Artificial sequence
  Flexible linker for modified HIV env gp140 for expression in plant cells
ggcggtggaggctcaggtggaggcggatcc

SEQ ID NO: 28 - DNA, 32 bp, Artificial sequence
  Flexible linker for modified HIV env gp140 for expression in mammalian cells
ggcggtggaggctcaggtggaggcggatccgc

SEQ ID NO: 29 - DNA, 30 bp, Artificial sequence; identical to SEQ ID NO: 27
  Flexible linker for modified Marburg viral glycoprotein for expression in plant cells

SEQ ID NO: 30 - DNA, 30 bp, Artificial sequence; identical to SEQ ID NO: 27
  Flexible linker for modified Marburg viral glycoprotein for expression in mammalian cells

SEQ ID NO: 31 - protein, 10 aa, Artificial sequence
  Amino acid sequence of the flexible linker
GGGGSGGGGS

SEQ ID NO: 32 - DNA, 2689 bp, Artificial sequence
  Modified Epstein-Barr virus gp350
aagcttccaccatggagtggtcatggatttttctgtttctgctgtctgggactgccggagtgcatagcgctgaggctgctctgctggtctgtcagtataccatccagagcctgatccacctgacaggcgaggaccccggcttctttaacgtggagatcccagagttccccttttaccctacatgcaacgtgtgcaccgccgacgtgaacgtgaccatcaatttcgatgtgggcggcaagaagcaccagctggacctggattttggccagctgacccctcacacaaaggccgtgtatcagccaagaggcgccttcggcggaagcgagaacgcaaccaatctgtttctgctggagctgctgggcgcaggagagctggccctgaccatgaggtccaagaagctgccaatcaacgtgaccacaggcgaggagcagcaggtgtccctggagtctgtggacgtgtacttccaggacgtgttcggcaccatgtggtgccaccacgccgagatgcagaatcccgtgtacctgatccctgagacagtgccatatatcaagtgggacaactgtaatagcaccaacatcacagcagtggtgagagcacagggcctggacgtgaccctgccactgagcctgcctacatccgcccaggatagcaacttctccgtgaagaccgagatgctgggcaatgagatcgacatcgagtgcatcatggaggatggcgagatctcccaggtgctgcctggcgataacaagtttaatatcacctgttctggatacgagagccacgtgccttccggaggcatcctgacctctacaagcccagtggcaacaccaatccctggaaccggctacgcctatagcctgcggctgaccccaagacccgtgtctaggttcctgggcaacaatagcatcctgtacgtgttttattccggaaacggaccaaaggcctctggaggcgactattgcatccagagcaatatcgtgttctccgacgagatccccgcctctcaggatatgcctaccaacaccacagacatcacatacgtgggcgataatgccacctatagcgtgcccatggtgacatctgaggacgccaacagccctaatgtgaccgtgacagccttctgggcctggccaaacaataccgagacagacttcaagtgcaagtggaccctgacatctggcacccccagcggctgtgagaacatctccggcgccttcgcctctaataggacatttgatatcaccgtgtccggcctgggcacagcccctaagaccctgatcatcacccgcacagccaccaacgccaccacaaccacacacaaagtgatcttcagcaaggcccctaggtccaccacaacctctccaaccctgaacacaaccggctttgccgacccaaatacaaccacaggcctgccaagctccacccacgtgccaaccaacctgacagcccctgcctctaccggcccaacagtgagcaccgccgatgtgacatcccctaccccagcaggaaccacatctggagcaagccccgtgactccatccccttctccatgggacaatggcacagagtctaaggcccccgatatgacatctagcaccagccctgtgaccacacccacccctaacgccacatctccaacccccgccgtgaccacacctaccccaaatgccacaagcccaacccctgcagtgaccacaccaaccccaaacgccacatcccccaccctgggcaagacatcccctacctctgccgtcactaccccaaccccaaatgccacatccccaaccctgggcaagacaagccccacctccgccgtgaccacaccaactccaaacgccacatctcctaccctgggcaagacatctccaaccagcgccgtcactacaccaaccccaaatgcaacaggcccaaccgtgggagagacaagccctcaggccaacgccacaaatcacaccctgggcggcacaagcccaaccccagtggtgacctcccagcccaagaacgccacatctgccgtgaccacaggccagcacaatatcacatcctctagcacctcctctatgagcctgcgcccaagctccaaccccgagacactgagcccttccacctctgacaatagcacatcccacatgccactgctgaccagcgcccacccaacaggcggagagaacatcacacaggtgaccccagcctctatcagcacccaccacgtgtccacatctagcccagcacctcggccaggaaccacatcccaggcctctggaccaggcaattcctctacaagcaccaagcccggcgaagtgaacgtgacaaagggaaccccacctcagaatgccaccagcccacaggcaccatccggacagaagacagcagtgccaacagtgaccagcacaggcggcaaggccaactccaccacaggcggcaagcacaccacaggccacggagcacggacctccacagagcctaccacagactacggcggcgattctaccacaccccggcctagatacaatgccaccacatatctgccaccaagcaccagctccaagctgaggccccgctggaccttcacatcccctccagtgaccacagcccaggcaaccgtcccagtcccccccacctcacagccaagattttccaacctgcatatgcatcatcaccatcaccattgatttttctgaattctcga

SEQ ID NO: 33 - protein, 886 aa, Artificial sequence
  Modified Epstein-Barr virus gp350
MEWSWIFLFLLSGTAGVHSAEAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPRSTTTSPTLNTTGFADPNTTTGLPSSTHVPTNLTAPASTGPTVSTADVTSPTPAGTTSGASPVTPSPSPWDNGTESKAPDMTSSTSPVTTPTPNATSPTPAVTTPTPNATSPTPAVTTPTPNATSPTLGKTSPTSAVTTPTPNATSPTLGKTSPTSAVTTPTPNATSPTLGKTSPTSAVTTPTPNATGPTVGETSPQANATNHTLGGTSPTPVVTSQPKNATSAVTTGQHNITSSSTSSMSLRPSSNPETLSPSTSDNSTSHMPLLTSAHPTGGENITQVTPASISTHHVSTSSPAPRPGTTSQASGPGNSSTSTKPGEVNVTKGTPPQNATSPQAPSGQKTAVPTVTSTGGKANSTTGGKHTTGHGARTSTEPTTDYGGDSTTPRPRYNATTYLPPSTSSKLRPRWTFTSPPVTTAQATVPVPPTSQPRFSNLHMHHHHHH

SEQ ID NO: 34 - DNA, 1964 bp, Artificial sequence
  Modified SOSIP.664
accggtatggatgctatgaaaagggggctgtgctgcgtcctgctgctgtgcggggctgtcttcgtgtcaccaagcgggctgtgggtcactgtctactatggcgtgcctgtctggagagaggccaagaccacactgttctgcgcttccgatgcaaagtcttacgaaaaagaggtgcacaacgtctgggccacacatgcttgcgtgccaactgaccccaaccctcaggaactggtgctgaagaatgtcaccgagaactttaatatgtggaaaaatgacatggtggatcagatgcacgaggatatcattagtctgtgggaccagtcactgaagccctgcgtgaaactgacacctctgtgcgtcactctgaactgtagcgatgcaaaggtgaacattaatgccacatacaatggcactcgcgaggaaatcaaaaactgttccttcaatgcaactaccgaactgagggacaagaagaagaaggagtacgccctgttttatcgcctggacatcgtgcccctgaacaaggaagggaacaataacagtgagtatcggctgattaactgcaataccagcgtgattacccaggcctgtcctaaagtcaccttcgatccaattcccatccactactgcgcaccagccggatatgctattctgaagtgtaacaacaaaacttttaacgggaccggaccctgcaataacgtgtctacagtccagtgtactcatggcatcaagcctgtggtctcaacccagctgctgctgaatgggagcctggccgaggaagagatcattatcagaagcgagaacctgaccgacaatgtgaagacaattatcgtccacctgaacgaatccgtggagattaattgcaccaggccaaacaacaacacacgaaaatctattcggatcggaccaggacagaccttctacgcaacaggggacattatcggagatatcaggcaggctcattgtaacatttctgaaatcaagtgggagaaaaccctgcagcgcgtgagtgaaaagctgcgagagcacttcaacaaaacaatcatctttaatcagagctccggcggggacctggaaatcacaactcattcattcaactgcggaggcgagttcttttactgtaacactagcgatctgttctttaataagacctttgacgagacctattccacaggctcaaacagcactaattctaccattacactgccatgccgaatcaaacagattatcaacatgtggcaggaagtgggccgggcaatgtatgccagccccattgccggagagatcacctgtaagtccaatatcactggactgctgctgaccagagatgggggaggcaacaattctactgaagagacctttaggcccgggggaggcaacatgagagacaattggaggagcgaactgtacaagtataaagtggtcgaggtgaagcctctgggaatcgcaccaaccgagtgccggagaagggtggtccagcgccgacggagaaggcgcgctgtggtcggactgggagcagtgttcctggggtttctgggaactgctggcagcaccatgggagccgcttccattactctgaccgtgcaggcacgccagctgctgtctggcatcgtccagcagcagagtaacctgctgcgggctcctgaagcacagcagcatatgctgcagctgaccgtgtgggggattaagcagctgcaggcccgggtcctggctatcgagagatacctgaaggatcagcagctgctggggatgtggggatgcagtggcaaactgatttgctgtacaaacgtgtactggaacagcagctggtccaacaagacatataatgaaatctgggacaacatgacttggatgcagtgggaccgcgagatcgataactacacagacactatctataaactgctggaagtctcacagaaacagcaggagtcaaatgaaaaggacctgctggcactggattaactcga

SEQ ID NO: 35 - protein, 794 aa, Artificial sequence
  Modified SOSIP.664
MELRPWLLWVVAATGTLVLLAADAQGQKVFTNTWAVRIPGGPAVANSVARKHGFLNLGQIFGDYYHFWHRGVTKRSLSPHRPRHSRLQREPQVQWLEQQVAKRRTKRDVYQEPTDPKFPQQWYLSGVTQRDLNVKAAWAQGYTGHGIVVSILDDGIEKNHPDLAGNYDPGASFDVNDQDPDPQPRYTQMNDNRHGTRCAGEVAAVANNGVCGVGVAYNARIGGVRMLDGEVTDAVEARSLGLNPNHIHIYSASWGPEDDGKTVDGPARLAEEAFFRGVSQGRGGLGSIFVWASGNGGREHDSCNCDGYTNSIYTLSISSATQFGNVPWYSEACSSTLATTYSSGNQNEKQIVTTDLRQKCTESHTGTSASAPLAAGIIALTLEANKNLTWRDMQHLVVQTSKPAHLNANDWATNGVGRKVSHSYGYGLLDAGAMVALAQNWTTVAPQRKCIIDILTEPKDIGKRLEVRKTVTACLGEPNHITRLEHAQARLTLSYNRRGDLAIHLVSPMGTRSTLLAARPHDYSADGFNDWAFMTTHSWDEDPSGEWVLEIENTSEANNYGTLTKFTLVLYGTAPEGLPVPPESSGCKTLTSSQACVVCEEGFSLHQKSCVQHCPPGFAPQVLDTHYSTENDVETIRASVCAPCHASCATCQGPALTDCLSCPSHASLDPVEQTCSRQSQSSRESPPQQQPPRLPPEVEAGQRLRAGLLPSHLPEVVAGLSCAFIVLVFVTVFLVLQLRSGFSFRGVKVYTMDRGLISYKGLPPEAWQEECPSDSEEDEGRGERTAFIKDQSAL

SEQ ID NO: 36 - DNA, 3830 bp, Artificial Sequence
  SARS-CoV-2
ccaccatggatgctatgaaaagaggactgtgctgcgtgctgctgctgtgcggagccgtgtttgtgagccccgtgaacctgactaccagaactcagctgccccctgcctataccaatagcttcacaaggggcgtgtactatcctgacaaggtgtttcgcagctccgtgctgcactccacacaggatctgtttctgccattcttttctaacgtgacctggttccacgccatccacgtgtccggcaccaatggcacaaagaggttcgacaatccagtgctgccctttaacgatggcgtgtacttcgcctccaccgagaagtctaacatcatccgcggctggatctttggcaccacactggacagcaagacacagtccctgctgatcgtgaacaatgccaccaacgtggtcatcaaggtgtgcgagttccagttttgtaatgatcccttcctgggcgtgtactatcacaagaacaataagtcttggatggagagcgagtttagggtgtattctagcgccaacaattgcacatttgagtacgtgagccagcctttcctgatggacctggagggcaagcagggcaatttcaagaacctgcgggagttcgtgtttaagaatatcgatggctacttcaagatctactctaagcacacccccatcaacctggtgcgggacctgcctcagggcttcagcgccctggagcctctggtggatctgccaatcggcatcaacatcaccaggtttcagacactgctggccctgcaccgctcctacctgacaccaggcgactcctctagcggatggaccgcaggagcagcagcctactatgtgggctatctgcagcctcggaccttcctgctgaagtacaacgagaatggcaccatcacagacgcagtggattgcgcactggaccccctgtctgagacaaagtgtacactgaagagctttaccgtggagaagggcatctatcagacaagcaatttccgggtgcagccaaccgagtccatcgtgagatttccaaatatcacaaacctgtgcccctttggcgaggtgttcaacgccaccagattcgccagcgtgtacgcctggaataggaagcgcatctctaactgcgtggccgactatagcgtgctgtacaactccgcctctttcagcacctttaagtgctatggcgtgtcccccacaaagctgaatgacctgtgctttaccaacgtgtacgccgattctttcgtgatcaggggcgacgaggtgcgccagatcgcacctggacagacaggcaagatcgccgactacaattataagctgccagacgatttcaccggctgcgtgatcgcctggaactctaacaatctggatagcaaagtgggcggcaactacaattatctgtaccggctgtttagaaagtccaatctgaagcccttcgagcgggacatctccacagagatctaccaggccggctctaccccttgcaatggcgtggagggctttaactgttatttcccactgcagtcttacggcttccagcccaccaacggcgtgggctatcagccttacagagtggtggtgctgagctttgagctgctgcacgcaccagcaacagtgtgcggacctaagaagtccaccaatctggtgaagaacaagtgcgtgaacttcaacttcaacggcctgaccggcacaggcgtgctgaccgagagcaacaagaagttcctgccatttcagcagttcggccgggacatcgcagataccacagacgccgtgcgggacccccagaccctggagatcctggatatcacaccctgctccttcggcggcgtgtctgtgatcacacccggcaccaatacatccaaccaggtggccgtgctgtatcaggacgtgaattgtaccgaggtgcctgtggccatccacgccgatcagctgaccccaacatggagggtgtacagcaccggctccaacgtgttccagacacgcgccggatgcctgatcggagcagagcacgtgaacaattcttatgagtgcgacatcccaatcggcgccggcatctgtgcctcctaccagacccagacaaactctccacggagaaggcgccggagaagcgtggcatcccagtctatcatcgcctataccatgtccctgggcgccgagaacagcgtggcctactctaacaatagcatcgccatcccaaccaacttcacaatcagcgtgaccacagagatcctgcccgtgagcatgaccaagacatccgtggactgcacaatgtatatctgtggcgattccaccgagtgctctaacctgctgctgcagtacggctccttttgtacccagctgaatagggccctgacaggcatcgcagtggagcaggataagaacacacaggaggtgttcgcccaggtgaagcagatctacaagaccccacccatcaaggactttggcggcttcaacttcagccagatcctgcccgatccttccaagccttctaagaggagctttatcgaggacctgctgttcaacaaggtgaccctggccgatgccggcttcatcaagcagtatggcgattgcctgggcgacatcgcagcccgcgacctgatctgtgcccagaagtttaatggcctgaccgtgctgcctccactgctgacagatgagatgatcgcccagtacacatctgccctgctggcaggaaccatcacaagcggatggaccttcggcgcaggagccgccctgcagatcccctttgccatgcagatggcctatcgcttcaacggcatcggcgtgacccagaatgtgctgtacgagaaccagaagctgatcgccaatcagtttaacagcgccatcggcaagatccaggacagcctgtcctctacagcctccgccctgggcaagctgcaggatgtggtgaatcagaacgcccaggccctgaataccctggtgaagcagctgagctccaacttcggcgccatctctagcgtgctgaatgatatcctgtccaggctggacaaggtggaggccgaggtgcagatcgaccggctgatcacaggcagactgcagtctctgcagacctacgtgacacagcagctgatcagggcagcagagatcagggcaagcgccaatctggcagcaaccaagatgagcgagtgcgtgctgggacagtccaagagggtggacttttgtggcaagggctatcacctgatgagcttcccacagtccgccccacacggagtggtgtttctgcacgtgacctacgtgcccgcccaggagaagaacttcaccacagcccctgccatctgccacgatggcaaggcccactttccacgggagggcgtgttcgtgtccaacggcacccactggtttgtgacacagagaaatttctacgagccccagatcatcaccacagacaataccttcgtgagcggcaactgtgacgtggtcatcggcatcgtgaacaataccgtgtatgatcctctgcagccagagctggacagctttaaggaggagctggataagtacttcaagaatcacacctcccccgacgtggatctgggcgacatctctggcatcaatgccagcgtggtgaacatccagaaggagatcgacagactgaacgaggtggccaagaatctgaacgagagcctgatcgatctgcaggagctgggcaagtatgagcagtacatcaagtggcctggatccggctccggctctccaggctccggatatatcccagaggcacctcgggacggacaggcctacgtgagaaaggatggcgagtgggtgctgctgagcaccttcctgggagggagcggcggcagcgggggcagcgggcaccatcaccaccaccactga

SEQ ID NO: 37 - protein, 1274 aa, Artificial Sequence
  SARS-CoV-2
MDAMKRGLCCVLLLCGAVFVSPVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRRRRRSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGSGSGSPGSGYIPEAPRDGQAYVRKDGEWVLLSTFLGGSGGSGGSGHHHHHH

SEQ ID NO: 38 - DNA, 3806 bp, Artificial Sequence
  SARS-CoV-2
ccaccatgtttgtctttctggtcctgctgcccctggtctcttcacagtgcgtcaatctgactacacgaactcagctgcctcctgcctatactaattccttcacccggggcgtgtactatccagacaaggtgtttagaagctccgtgctgcactccacacaggatctgtttctgcccttcttttctaacgtgacctggttccacgccatccacgtgagcggcaccaatggcacaaagcggttcgacaatcctgtgctgccatttaacgatggcgtgtacttcgcctccaccgagaagtctaacatcatcagaggctggatctttggcaccacactggacagcaagacacagtccctgctgatcgtgaacaatgccaccaacgtggtgatcaaggtgtgcgagttccagttttgtaatgatcctttcctgggcgtgtactatcacaagaacaataagtcttggatggagagcgagtttagagtgtattctagcgccaacaattgcacatttgagtacgtgtcccagccattcctgatggacctggagggcaagcagggcaatttcaagaacctgagggagttcgtgtttaagaatatcgatggctacttcaagatctacagcaagcacaccccaatcaacctggtgcgcgacctgccacagggattctccgccctggagccactggtggatctgcctatcggcatcaacatcacccggtttcagacactgctggccctgcacagaagctacctgacacctggcgactcctctagcggatggaccgcaggagcagcagcctactatgtgggctatctgcagccacggaccttcctgctgaagtacaacgagaatggcaccatcacagacgcagtggattgcgccctggaccccctgtctgagacaaagtgtacactgaagagctttaccgtggagaagggcatctatcagacaagcaatttcagggtgcagcccaccgagtccatcgtgcgctttcccaatatcacaaacctgtgcccttttggcgaggtgttcaacgccaccagattcgccagcgtgtacgcctggaatcggaagagaatctccaactgcgtggccgactattctgtgctgtacaacagcgcctccttctctacctttaagtgctatggcgtgtctcctacaaagctgaatgatctgtgctttaccaacgtgtacgccgatagcttcgtgatcaggggagacgaagtgagacagatcgcaccaggacagacaggcaagatcgcagactacaattataagctgcctgacgatttcaccggctgcgtgatcgcctggaactctaacaatctggatagcaaagtgggcggcaactacaattatctgtacaggctgtttcgcaagtccaatctgaagcctttcgagagggacatctccacagagatctaccaggccggctctaccccatgcaatggcgtggagggctttaactgttatttccctctgcagtcttacggcttccagccaacaaacggcgtgggctatcagccctaccgcgtggtggtgctgtcctttgagctgctgcacgcacctgcaacagtgtgcggaccaaagaagtctaccaatctggtgaagaacaagtgcgtgaacttcaacttcaacggcctgaccggcacaggcgtgctgaccgagtccaacaagaagttcctgccctttcagcagttcggcagggacatcgcagataccacagacgccgtgcgcgaccctcagaccctggagatcctggatatcacaccatgctccttcggcggcgtgtctgtgatcacacctggcaccaatacaagcaaccaggtggccgtgctgtatcaggacgtgaattgtaccgaggtgcccgtggcaatccacgcagatcagctgacccctacatggcgggtgtactctaccggcagcaacgtgttccagacaagagccggatgcctgatcggagcagagcacgtgaacaatagctatgagtgcgacatccccatcggcgccggcatctgtgcctcctaccagacccagacaaactcccctggatccgcctcctctgtggcaagccagtccatcatcgcctataccatgtccctgggcgccgagaacagcgtggcctacagcaacaattccatcgccatccctaccaacttcacaatctccgtgaccacagagatcctgccagtgagcatgaccaagacatccgtggactgcacaatgtatatctgtggcgattccaccgagtgctctaacctgctgctgcagtacggcagcttttgtacccagctgaatagggccctgacaggcatcgcagtggagcaggataagaacacacaggaggtgttcgcccaggtgaagcagatctacaagaccccccctatcaaggactttggcggcttcaacttcagccagatcctgcccgatccttctaagccaagcaagcggtcccccatcgaggacctgctgtttaacaaggtgaccctggccgatgccggcttcatcaagcagtatggagattgcctgggagacatcgcagcccgggacctgatctgtgcccagaagttcaatggcctgaccgtgctgccacccctgctgacagatgagatgatcgcccagtacacatctgccctgctggcaggaaccatcacaagcggatggacctttggcgcaggaccagccctgcagatcccatttcccatgcagatggcctatcgcttcaacggcatcggcgtgacccagaatgtgctgtacgagaaccagaagctgatcgccaatcagttcaactccgccatcggcaagatccaggacagcctgagctccacaccatccgccctgggaaagctgcaggatgtggtgaatcagaacgcccaggccctgaataccctggtgaagcagctgtctagcaactttggcgccatctcctctgtgctgaatgatatcctgagcaggctggaccctccagaggcagaggtgcagatcgacaggctgatcacaggccgcctgcagagcctgcagacctatgtgacacagcagctgatcagggcagcagagatcagagcatccgccaatctggccgccaccaagatgagcgagtgcgtgctgggacagtccaagagggtggacttttgtggcaagggctatcacctgatgagcttccctcagtccgccccacacggagtggtgtttctgcacgtgacctacgtgccagcccaggagaagaacttcaccacagcaccagcaatctgccacgatggaaaggcacactttcccagggagggcgtgttcgtgtctaacggcacccactggtttgtgacacagcgcaatttctacgagcctcagatcatcaccacagacaatacattcgtgagcggcaactgtgatgtggtgatcggcatcgtgaacaataccgtgtatgatcccctgcagcctgagctggactcttttaaggaggagctggataagtacttcaagaatcacaccagccccgacgtggatctgggcgacatctctggcatcaatgccagcgtggtgaacatccagaaggagatcgaccggctgaacgaggtggccaagaatctgaacgagtccctgatcgatctgcaggagctgggcaagtatgagcagggctctggatacatcccagaggcacccagggacggacaggcctacgtgcgcaaggatggcgagtgggtgctgctgtctacctttctgggcagaagcctggaggtgctgttccagggaccaggacaccatcatcaccaccaccaccactctgcctggagccacccccagttcgagaag

SEQ ID NO: 39 - protein, 1288 aa, Artificial Sequence
  SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGHHHHHHHHSAWSHPQFEKGGGSGGGGSGGSAWSHPQFEK

SEQ ID NO: 40 - DNA, 1496 bp, Artificial Sequence
  Ebola virus
ccaccatggatgccatgaagaggggcctgtgctgcgtcctgctgctgtgcggggccgtgttcgtgtccccctccattcccctcggcgtgatccacaacagcaccctgcaggtgagcgatgtggataagctggtgtgccgggacaagttgagcagcactaaccagctgcggagcgtcggcctgaacctggaggggaacggcgtggccacagatgtgccaagcgccacaaagaggtgggggttcaggagcggcgtgccccctaaggtggtgaactacgaggccggggagtgggccgagaactgctacaacctggagatcaagaagcccgacgggagcgagtgcctgccagccgcccccgacggcattaggggctttcctaggtgcaggtacgtgcacaaggtgagcgggaccggcccttgcgccggggacttcgcctttcacaaggagggcgccttcttcctgtacgatagactggccagcaccgtgatctaccggggcacaaccttcgccgagggggtggtcgcctttctcatcctgccccaggccaagaaggatttcttctccagccaccccctgcgggagccagtgaacgccaccgaggaccccagctccggctactactccaccacaatccggtaccaggctactggcttcgggaccaacgagactgagtacctgttcgaggtcgacaacctgacatacgtccagctcgaaagccggttcaccccccagttcctgctgcagctgaacgagactatctacaccagcgggaagaggtctaacaccacagggaagctgatctggaaggtgaaccccgagattgatacaaccatcggggagtgggccttttgggagaccaagaagaacctgacacggaagattcggagcgaggagctgagcttcaccgtggtctccaacggcgccaagaacattagcggcaagctggggctcatcaccaacacaatcgctggcgtcgccggcctgatcaccggggggaggcggacacggcgggaggccattgtgaacgcccagccaaagtgcaaccctaacctccactactggacaacacaggacgagggcgccgccatcgggctggcctggatcccttacttcggccccgccgccgagggcatctacattgagggcctgatgcacaaccaggacggcctgatctgcgggctgaggcagctggccaacgagacaacccaggccctgcagctgtttctgcgggccacacccgagctgcggacattttccattctgaacaggttcgccatcgatttcctgctgcagcggtggggcgggacctgccacattctcggccctgattgctgcattgagccccacgactggaccaagaacatcacagataagatcgaccagatcatccacgatttcgtggataagacactgcccgaccagggggataacgacaactggtggacaggcgggagcggcggcagcggcggcagcggccaccaccatcaccaccactga

SEQ ID NO: 41 - protein, 496 aa, Artificial Sequence
  Ebola Virus
MDAMKRGLCCVLLLCGAVFVSPSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVSNGAKNISGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATPELRTFSILNRFAIDFLLQRWGGTCHILGPDCCIEPHDWTKNITDKIDQIIHDFVDKTLPDQGDNDNWWTGGSGGSGGSGHHHHHH

SEQ ID NO: 42 - DNA, 1610 bp, Artificial Sequence
  Nipah virus
ccaccatggtggtgatcctggacaagcggtgctactgtaacctgctgatcctgatcctgatgatctccgagtgctctgtgggcatcctgcactacgagaagctgtccaagatcggcctggtgaagggcgtgacccggaagtataagatcaagtctaatcctctgacaaaggatatcgtgatcaagatgatcccaaacgtgagcaatatgtcccagtgtaccggctccgtgatggagaactacaagacccgcctgaatggcatcctgacaccaatcaagggcgccctggagatctataagaacaatacacacgactgcgtgggcgatgtgcggctggcaggcgtgtgcatggcaggagtggcaatcggaatcgcaaccgcagcacagatcacagcaggagtggccctgtatgaggccatgaagaacgccgacaacatcaataagctgaagagctccatcgagagcaccaatgaggccgtggtgaagctgcaggagaccgccgagaagacagtgtacgtgttcacagccctgcaggactatatcaacaccaatctggtgcccacaatcgataagatcccttgcaagcagaccgagctgagcctggacctggccctgtccaagtacctgtctgatctgctgttcgtgtttggccctaacctgcaggatccagtgtccaattctatgacaatccaggccatctcccaggccttcggcggcaactacgagaccctgctgagaacactgggctatgccaccgaggactttgacgatctgctggagagcgattccatcacaggccagatcatctatgtggacctgtctagctactatatcatcgtgagggtgtacttccccatcctgaccgagatccagcaggcctatatccaggagctgctgcccgtgagcttcaacaatgataacagcgagtggatctccatcgtgccaaacttcatcctggtgcggaacaccctgatctctaatatcgagatcggcttttgcctgatcacaaagagaagcgtgatctgtaaccaggactacgccacccccatgacaaacaatatgagggagtgcctgaccggcagcacagagaagtgtccaagggagctggtggtgtcctctcacgtgccaaggttcgcactgagcaacggcgtgctgtttgccaattgcatctccgtgacctgccagtgtcagaccacaggcagagccatctctcagagcggcgagcagaccctgctgatgatcgataacaccacatgtcccacagccgtgctgggcaatgtgatcatctccctgggcaagtacctgggctccgtgaactataattctgagggaatcgcaatcggaccccccgtgttcaccgacaaggtggatatcagctcccagatctctagcatgaaccagtctctgcagcagagcaaggactacatcaaggaggcccagcggctgctggatacagtgaatccttctctgggcagcggctacatcccagaggcaccccgggacggacaggcctatgtgagaaaggatggcgagtgggtgctgctgagcacctttctgggatccggaggatctggaggaagcggacaccatcatcaccaccaccaccactga

SEQ ID NO: 43 - protein, 534 aa, Artificial Sequence
  Nipah virus
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDCVGDVRLAGVCMAGVAIGIATAAQITAGVALYEAMKNADNINKLKSSIESTNEAVVKLQETAEKTVYVFTALQDYINTNLVPTIDKIPCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLGSGYIPEAPRDGQAYVRKDGEWVLLSTFLGSGGSGGSGHHHHHHHH

SEQ ID NO: 44 - DNA, 1238 bp, Artificial Sequence
  Lujo virus
ccaccatggggcagattgtcgctgtcttccaggctattcccgagattctgaacgaggctatcaacatcgtcatcattgtcatcattatgtttaccctgatcaagggcgtgtttaatctgtacaagtccggcctgtttcagctggtcatcttcctgctgctgtgcggcaagaggtgtgatagctccctgctgtctggctttaacctggagacagtgcacttcaatatgagcctgctgtctagcatccccatggtgtccgagcagcagcactgtatccagcacaatcactcctctatcaccttttctctgctgacaaacaagagcgacctggagaagtgcaatttcaccaggctgcaggccgtggatcgcgtgatcttcgacctgtttagggagttccaccaccgcgtgggcgattttcctgtgaccagcgacctgaagtgtagccacaacacatcctacagagtgatcgagtatgaggtgaccaaggagtctctgccaagactgcaggaggccgtgagcacactgtttcccgatctgcacctgtccgaggaccgcttcctgcagatccaggcccacgacgataagaactgtaccggcctgcacccactgaattacctgaggctgctgaaggagaactccgagacacactataaggtgcgcaagctgatgaagctgttccagtggagcctgagcgatgagacaggcagccccctgcctggaggacactgcctggagcggtggctgatctttgccagcgatatcaagtgcttcgacaacgccgccatcgccaagtgtaataaggagcacgatgaggagttttgcgacatgctgcggctgttcgattacaacaaggccagcatcgccaagctgagaggcgaggccagctcctctatcaacctgctgtccggcaggatcaatgccatcatctctgacacactgctgatgcggagctccctgaagagactgatgggcatcccttactgtaattataccaagttttggtacctgaaccacacaaagctgggcatccactccctgccacggtgctggctggtgtccaacggctcttatctgaatgagacaaagttcacacacgacatggaggatgaggccgacaagctgctgaccgagatgctgaagaaggagtatgtgcggagacaggagaagacacccatcaccctgatggacattggaagcggaggcagcggcggatccggacaccaccaccaccaccactga

SEQ ID NO: 45 - protein, 410 aa, Artificial Sequence
  Lujo virus
MGQIVAVFQAIPEILNEAINIVIIVIIMFTLIKGVFNLYKSGLFQLVIFLLLCGKRCDSSLLSGFNLETVHFNMSLLSSIPMVSEQQHCIQHNHSSITFSLLTNKSDLEKCNFTRLQAVDRVIFDLFREFHHRVGDFPVTSDLKCSHNTSYRVIEYEVTKESLPRLQEAVSTLFPDLHLSEDRFLQIQAHDDKNCTGLHPLNYLRLLKENSETHYKVRKLMKLFQWSLSDETGSPLPGGHCLERWLIFASDIKCFDNAAIAKCNKEHDEEFCDMLRLFDYNKASIAKLRGEASSSINLLSGRINAIISDTLLMRSSLKRLMGIPYCNYTKFWYLNHTKLGIHSLPRCWLVSNGSYLNETKFTHDMEDEADKLLTEMLKKEYVRRQEKTPITLMDIGSGGSGGSGHHHHHH